(19) United States
(12) Patent Application Publication
(10) Pub. No.: US 2023/0287391 A1
(43) Pub. Date: Sep. 14, 2023

(54) KRAB FUSION REPRESSORS AND METHODS AND COMPOSITIONS FOR REPRESSING GENE EXPRESSION

(71) Applicant: THE GOVERNING COUNCIL OF THE UNIVERSITY OF TORONTO, Toronto (CA)

(72) Inventors: Mikko Taipale, Toronto (CA); Nader Alerasool, Toronto (CA)

(21) Appl. No.: 18/020,991

(22) PCT Filed: Aug. 14, 2021
(86) PCT No.: PCT/CA2021/051121

§ 371 Date: Feb. 13, 2023

Related U.S. Application Data
(60) Provisional application No. 63/065,953, filed on Aug. 14, 2020.

(51) Int. Cl. C12N 15/10; C12N 15/11; C12N 9/22

(52) U.S. Cl. CPC C12N 15/1051; C12N 15/11; C12N 15/111; C12N 9/22; C12N 2310/20; C12N 2310/14

(57) ABSTRACT

A heterologous transcriptional repressor comprising a DNA targeting domain, preferably a catalytically inactive DNA targeting protein such as a CRISPR-Cas protein, and a KRAB domain selected from the group consisting of ZIM3-KRAB, ZIM2-KRAB, ZNF554-KRAB, ZNF264-KRAB, ZNF324-KRAB, ZNF354A-KRAB, ZFP82-KRAB, and ZNF669-KRAB. Also provided herein are expression constructs, vectors, and cells encoding or expressing said transcriptional repressor, as well as systems and methods for transcriptional repression of a target gene, and compositions, kits and reagents employed in the making and use thereof.

### RELATED FAMILY MEMBERS

This is a PCT application which claims the benefit of priority of U.S. Patent Application Ser. No. 63/065,953 filed Aug. 14, 2020, which is incorporated herein by reference.

### INCORPORATION OF SEQUENCE LISTING

A computer readable form of the Sequence Listing “2223-P61944PC00_SequenceListing” (56,320 bytes), created on Aug. 13, 2021, is herein incorporated by reference.

### FIELD

The present disclosure relates to reagents and methods for transcriptional repression and in particular to the use of heterologous KRAB domain in transcriptional repressors for targeted transcriptional repression.

### INTRODUCTION

The catalytically inactive dCas9 fused to a Krüppel-associated box (KRAB) transcriptional repression domain has been widely adopted as a genetic screening tool known as CRISPRi [1-4]. CRISPRi lacks the non-specific cellular toxicity of Cas9 caused by formation of DNA double-strand breaks, allows for silencing of non-coding RNAs, and enables discovery of distal regulatory regions [1,5,6]. However, in many cases CRISPRi does not work as robustly as knockout screens based on active Cas9 (CRISPR-KO). For example, CRISPRi is more sensitive to gRNA selection than CRISPR-KO [7]. Moreover, even when CRISPRi works, gene silencing is often only partial, limiting the utility of the method [8]. These challenges have been partially addressed by designing more efficient gRNA libraries [7,9], on one hand, and trying different repressor constructs fused to dCas9 [9-11], on the other. All these approaches use the well-characterized KRAB domain from KOX1 (ZNF10), a potent transcriptional repressor [10]. Recently, a systematic search for a better repressor yielded the bipartite repressor that consists of Methyl-CpG Binding Protein 2 (MeCP2) fused to KOX1 KRAB domain. KRAB-MeCP2-dCas9 repressor outperformed KOX1 KRAB-dCas9 in multiple assays [8].

### SUMMARY

Precisely controlled regulation of gene expression in cells, tissues, and organisms is one of the major challenges in tissue engineering, cell-based therapies, and gene therapy. Controlled gene expression in cells is achieved either by exogenously introducing cDNAs by transfection or viral transduction, or by introducing sequence-specific transcriptional regulators. These regulators include zinc-finger nucleases, tet-repressor and variants thereof, transcription activator like effectors (TALEs), or enzymatically inactive Cas9 (dCas9) which are fused to transcriptional repression domains to repress gene expression. Regulation of endogenous loci is a particularly attractive option as it circumvents the dramatic decrease in viral titer with larger cDNAs and enables silencing of gene expression in a tissue-specific manner.

Currently, only a few effector domains are used for transcriptional repression. The most commonly used domain for CRISPR inhibition (CRISPRi) is Kruppel-associated box (KRAB) domain of ZNF10 protein, although recently a bipartite system that uses ZNF10 KRAB together with methyl-CpG-binding protein 2 (MeCP2) was shown to be more effective in silencing multiple genes. Although CRISPRi is widely employed, it has two major limitations. First, most gRNAs do not work efficiently, thus requiring extensive testing of multiple gRNAs to identify functional ones. This is a major limitation in e.g. genome-wide CRISPRi screens. The second issue is that even when gRNAs work, the extent of transcriptional repression may be nonoptimal. That is, KRAB fusions only partially silence gene expression.

The inventors have identified protein domains that, when tethered to a promoter or 3′ UTR of a gene, leads to more complete repression of gene expression than with currently available systems. The inventors tested 57 different KRAB (Kruppel associated box) domains fused to enzymatically inactive dCas9 for their ability to silence an EGFP reporter driven by SV40 promoter. These KRAB domains showed a wide range of repressive ability from essentially no repression to almost complete repression (FIG. 5). Notably, KRAB domain from KOX1 did not repress the reporter very robustly. This is significant, because KOX1 KRAB is currently used in all CRISPRi-based platforms to regulate gene expression in human cells and wide adoption of CRISPRi has been hampered by its unpredictable nature. For example, many gRNAs used for CRISPRi do not work, and when they do work, they often repress transcription only partially. Therefore, many laboratories have tried to develop more potent platforms. The most recent one, dCas9-KOX1 KRAB-MeCP2 [8] works better than KOX1 KRAB domain as it uses two repressor domains in tandem. However, as described herein, other KRAB domains including the ZIM3 KRAB domain outperform this platform at multiple different loci (FIG. 6). Thus, the platform described herein facilitates robust repression of translation than existing systems. There are several applications where this system would be highly useful, including but not limited to: CRISPRi screening for identifying regulatory elements important for gene expression; CRISPRi silencing of noncoding transcripts; and silencing of large chromosomal domains to repress multiple genes at the same time. This would be advantageous for silencing e.g. microduplications implicated in human diseases. Furthermore, because KRAB domain is only ˜200 bp long, it facilitates more efficient packaging of e.g. adenoviral vectors compared to e.g. the KRAB-MeCP2 fusion.

This system described herein is not limited to dCas9 but can be coupled to other gene repressor targeting systems like engineered zinc fingers comprising a selected ZnF DNA binding domain, tet-repressor, or TALEs. TALE-KRAB based transcriptional repressor vectors have been used to knockdown multiple gene targets as described in Zhang et al, 2015 [22] and for tetracycline-reversible silencing of eukaryotic promoters [23].

Accordingly, an aspect is a heterologous transcriptional repressor comprising:
a DNA targeting domain, optionally a CRISPR-Cas protein, preferably an enzymatically inactive CRISPR-CAS 9 protein, zinc finger domain, tet-repressor or TALE;
and at least one KRAB domain selected from the group consisting of ZIM3-KRAB, ZIM2-KRAB, ZNF554-KRAB, ZNF264-KRAB, ZNF324-KRAB, ZNF354A-KRAB, ZFP82-KRAB, and ZNF669-KRAB.

Another aspect is an isolated nucleic acid encoding the transcriptional repressor, or an expression construct, vector or cell comprising said nucleic acid.

An aspect includes an expression construct comprising a nucleic acid described herein operably linked to one or more promoters and/or one or more transcription termination sites.

An aspect includes a vector comprising a nucleic acid or expression construct described herein, optionally wherein the vector is an adenoviral or lentiviral vector.

An aspect includes a cell comprising a transcriptional repressor, nucleic acid, expression construct, or vector described herein.

A further aspect includes a transcriptional repression system comprising:
the heterologous transcriptional repressor described herein a nucleic acid described herein, an expression construct described herein, a vector described herein or a cell described herein, wherein the DNA targeting domain comprises a CRISPR-Cas protein, and
at least one gRNA and/or an inducing agent.

An aspect includes a method of repressing transcription of a target gene in a cell, the method comprising: a) introducing into the cell a transcriptional repressor, nucleic acid, expression construct, or vector described herein; and b) culturing the cell under suitable conditions such that the at least one KRAB domain represses transcription of the target gene.

An aspect includes a screening method, the method comprising: a) introducing into a plurality of cells a transcriptional repressor, one or more nucleic acids, one or more expression constructs, or one or more vectors described herein, wherein the DNA targeting domain comprises a CRISPR-Cas protein; and a plurality of gRNAs; or introducing a plurality of gRNAs into a population of cells described herein wherein the DNA targeting domain comprises a CRISPR-Cas protein; b) culturing the plurality of cells such that the one or more gRNAs associate with the CRISPR-Cas protein and guides the transcriptional repressor to a CRISPR target site such that the at least one KRAB domain represses transcription of a target gene; c) optionally treating with an amount of a test drug or toxin; d) optionally culturing the plurality of cells for a period of time to allow for gRNA dropout or enrichment; and e) collecting the plurality of cells, or a subset thereof.

An aspect includes a composition comprising a transcriptional repressor, nucleic acid, expression construct, vector, or cell described herein.

An aspect includes a kit comprising a vial and a heterologous transcriptional repressor, nucleic acid, expression construct, vector, cell, or composition described herein and optionally one or more of: an inducing agent, a gRNA or a gRNA expression construct.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIG. 1a-h show identification of highly potent KRAB domains. a, Schematic of the two reporters used in assaying KRAB-domain activity. The Venn diagram indicates the number of KRAB domains assayed within HEK293T cells, K562 cells or in both cell lines. b, HEK293T reporter cell lines stably expressing gRNAs were infected with KRAB-dCas9 fusion constructs, and EGFP expression was analyzed 21 d later with flow cytometry. KOX1 KRAB-dCas9 and KOX1 KRAB-MeCP2-dCas9, which are used in current implementations of CRISPRi, are highlighted. c, Silencing results in K562 reporter cells. Assay was performed as in b except that the dCas9 constructs also expressed DsRed (KRAB-dCas9-P2A-DsRed). EGFP fluorescence is normalized to reporter cells expressing gRNA only, in b and c. d, Correlation between the repressive activity of KRAB domains and the number of TRIM28 peptides recovered by affinity purification—mass spectrometry with full-length KRAB zinc finger proteins (data from ref. 9). e, Correlation between the repressive activity of KRAB domains and their interaction with TRIM28 as measured by LUMIER assay in HEK293T cells. Interaction strength is shown as fold change over negative control bait (EGFP). The values shown are an average of two biological replicates. Spearman correlation was calculated from log10-transformed data without multiple hypothesis correction in d and e. f, ZIM3 KRAB and KOX1 KRAB were recruited to the SV40-EGFP reporter with an ABA-based dimerization system. g, EGFP silencing was induced by treating cells with 100 μM ABA. After 9 d, ABA was either washed off or the treatment was continued for another 11 d. EGFP expression was measured by flow cytometry and normalized to reporter cells expressing Nanoluc-PYL1. The values shown are an average of two biological replicates. Error bars denote standard deviation. h, EGFP silencing was induced by treating KRAB-PYL1 and ABI1-dCas9 expressing SV40-EGFP reporter cells with 100 μM ABA. After 40 days of ABA treatment, ABA was washed off and EGFP expression followed with flow cytometry for another 48 days. EGFP fluorescence was normalized to that of Firefly luciferase-dCas9 fusion similarly recruited to the reporter. The values shown are from a single biological replicate.

FIGS. 2a-e show benchmarking ZIM3 KRAB-dCas9 fusion in CRISPRi applications. a, dCas9 fusions were recruited to ERK1 and SEL1L promoters with a single gRNA for 7 d, and messenger RNA expression was quantified with RT-qPCR. Expression levels are normalized to that of HEK293T cells expressing no gRNA. A two-tailed Student's t-test with Bonferroni correction for multiple hypotheses was used to assess statistical significance. n, three independent lentiviral infections. b, Indicated dCas9 fusions were recruited to CD81 promoter and CD81 surface expression was measured by flow cytometry 7 d after infection. c, dCas9 fusions were recruited to HBEGF with five different gRNAs or as a pool. After 7 d, the sensitivity of cell lines to DTA was measured by serial dilution. Dotted lines indicate the sensitivity of HBEGF knockout cells (top) or cells with no gRNA. Right panel, half-maximal growth inhibition (GI50) curves were calculated for gRNA2. Data are presented as mean±s.d. (n, three treated wells for each concentration). GI50 values were calculated with GraphPad Prism using ‘log(inhibitor) versus response (three parameters)’ nonlinear fit. d, HEK293T cells stably expressing dCas9 fusions were infected with the genome-wide Dolcetto set A gRNA library and gRNA representation was measured after 21 d in culture. ROC curves calculated based on the depletion of gRNAs targeting gold-standard essential and nonessential genes. Average gRNA depletion was used for gene-level metrics. e, AUROC was calculated for each screen at the guide or gene level. To directly compare our results to a previous screen conducted in HEK293T cells, only the subset of genes that was targeted by both screens was analyzed.

FIG. 3 shows A) Expression of KRAB-dCas9 fusions and repressive activity. Expression was measured by western blotting; B) Correlation between KRAB silencing activity and TRIM28 peptides recovered by affinity purification of full-length KRAB domain proteins (data from BioPlex/Huttlin et al. 2018 and Imbeault et al. 2019); and C) Indicated dCas9 fusions were recruited to two endogenous promoters and gene expression measured by qRT-PCR. D) Expression level of TRIM28 in HEK293T, K562 and A375 cells.

FIGS. 4A-C show tethering KRAB domains to genomic loci to repress transcription. dCas9-KRAB fusions can be tethered to promoters (A), distal regulatory sites (B), or potentially distal non-regulatory sites (C) to repress transcription. In the case of distal nonregulatory sites, KRAB domains can induce the formation of heterochromatin that spreads to flanking regions.

FIG. 5 shows KRABs exhibit a range of transcriptional repression activity. dCas9-KRAB fusions were recruited to a SV40 promoter driving the expression of EGFP. Fluorescence was measured 21 days after infection with dCas9-KRAB fusions. Dashed line shows repression induced by dCas9 fused to Renilla luciferase (RLuc).

FIG. 6 shows dCas9 fused to the KRAB domain of ZIM3 is more effective in silencing gene expression than dCas9 fused to KRAB domain of KOX1 alone or to KOX1 KRAB and MeCP2. HEK293T cells were infected with gRNAs targeting ERK1, SEL1L, BLM, and MET promoters and with dCas9 fusions.

FIGS. 7A-C show ZIM3 KRAB domain represses transcription from 3′UTR of a EGFP reporter more efficiently than KOX1 KRAB. A) KRAB domain was recruited to dCas9 with abscisic acid based proximity induction system. B) Reporter gene in the AAVS1 locus in K562 cells. C) EGFP fluorescence of cells after treating them with 100 μM abscisic acid for 5 days or 14 days (or left untreated).

FIG. 8 shows RNA-seq analysis of a HEK293T SV40-EGFP reporter cell line expressing indicated dCas9 fusions targeted to the SV40 promoter. Differentially expressed transcripts (absolute log2 fold-change >0.5 and FDR <0.05) are shown as solid circles.

FIGS. 9A-C are a series of graphs and immunoblots. A, Correlation between the efficacy of KRAB-dCas9 fusions in the HEK293T reporter cell line and the K562 reporter cell line. Correlation was calculated using log₁₀-transformed values. B, Comparison of different KOX1 KRAB and ZIM3 KRAB domain constructs. Indicated KRAB-dCas9 fusions were recruited to the CD81 promoter (left) in A375 and HEK293T cells or to the SV40-EGFP reporter in HEK293T cells (right) and EGFP fluorescence was measured by flow cytometry. KOX1 (1-75) pLX311 is a lentiviral construct used in previous CRISPRi studies. The numbers in construct labels refer to the amino acids of KOX1 (Uniprot P21506-1) and ZIM3 (Q96PE6-1) included in the fusion. C, Expression level of dCas9 fusion proteins was assayed by western blotting with a Cas9-specific antibody.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

The terms “nucleic acid”, “oligonucleotide”, “primer” as used herein means two or more covalently linked nucleotides. Unless the context clearly indicates otherwise, the term generally includes, but is not limited to, deoxyribonucleic acid (DNA) and ribonucleic acid (RNA), which may be single-stranded (ss) or double stranded (ds). For example, the nucleic acid molecules or polynucleotides of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The term “oligonucleotide” as used herein generally refers to nucleic acids up to 200 base pairs in length and may be single-stranded or double-stranded. The sequences provided herein may be DNA sequences or RNA sequences, however it is to be understood that the provided sequences encompass both DNA and RNA, as well as the complementary RNA and DNA sequences, unless the context clearly indicates otherwise. For example, the sequence 5′-GAATCC-3′, is understood to include 5′-GAAUCC-3′,5′-GGATTC-3′, and 5′GGAUUC-3′.

The term “functional variant” as used herein includes modifications of the polypeptide sequences disclosed herein that perform substantially the same function as the polypeptide molecules disclosed herein in substantially the same way. For example, functional variants may include active fragments of the polypeptides described herein, for example an N- and/or C-terminal truncation which retain transcriptional repression activity and/or co-repressor (e.g. TRIM28) interaction. Functional variants may include variants having one or more substituted amino acids and/or which retain at least a minimal sequence identity to the unmodified sequence. For example, the functional variant may comprise substitutions of up to 1, 2, 3, or more amino acids for every ten amino acids. For example, the functional variant may comprise sequences having at least 80%, or at least 90%, or at least 95% sequence identity to the sequences disclosed herein. The functional variant may also comprise conservatively substituted amino acid sequences of the sequences disclosed herein. Substitutional amino acid variants are those in which at least one residue in the sequence has been removed and a different residue inserted in its place. An example of substitutional amino acid variants are conservative amino acid substitutions. Functional variants such as active fragments which retain transcriptional repression activity and/or co-repressor interaction can be identified for example using the methods described herein.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue or non-natural amino acid in place of a non-derivatized residue provided that such polypeptide displays the requisite activity.

The term “heterologous transcriptional repressor” or “transcriptional repressor described herein” as used herein means an engineered fusion protein or engineered multimer such as a dimer comprising a KRAB domain selected from ZIM3-KRAB, ZIM2-KRAB, ZNF554-KRAB, ZNF264-KRAB, ZNF324-KRAB, ZNF354A-KRAB, ZFP82-KRAB, and ZNF669-KRAB and functional variants thereof, and a DNA targeting domain.

The transcriptional repressor may further comprise one or more interaction components of an interaction system, which provides a functional interaction between the KRAB domain and/or DNA targeting domain and/or target DNA. The term “interaction component” is used herein to encompass one or more components of an interaction system, which together provide said functional interaction. The term “interaction system” as used herein is intended to encompass interaction components that permit covalent or non-covalent interactions, and/or constitutive or inducible interactions. Such interaction systems may include for example a peptide linker, optionally a protease-sensitive peptide linker; one or more dimer, trimer, or higher order multimerization components such as an interaction domain, optionally inducible dimer, trimer, or multimerization components, optionally an inducible interaction domain; and/or one or more components which can modulate subcellular localization of the transcriptional repressor. The interaction system can comprise two or more components.

The DNA targeting domain and the KRAB domain may be covalently linked, for example as domains of a single polypeptide (e.g. fusion protein), or may be linked by an interaction component such as an interaction domain for example, that interact under certain conditions (e.g. as a dimer). Accordingly, the heterologous transcriptional repressor may comprise a single polypeptide, or may comprise a first polypeptide comprising a DNA targeting domain and a first interaction component such as a dimer interaction domain, and a second polypeptide comprising a KRAB domain and a second interaction component such as a dimer interaction domain, wherein the first and second dimer interaction domain can interact, for example under certain conditions. Higher-order multimerization systems, such as the SunTag system (Tenenbaum et al., 2014), are also contemplated herein.

The interaction between the KRAB domain and/or DNA targeting domain and/or target DNA can be controlled using a variety of inducible interaction systems. For example, the KRAB domain and DNA targeting domain may be linked by a protease-sensitive linker such as a self-cleaving NS3 protease domain, which is stabilized in the presence of an NS3 inhibitor such as grazoprevir. In another example, localization of the DNA targeting domain and/or KRAB domain to the nucleus can be controlled by an interaction component such as a localization domain, for example tamoxifen-regulated nuclear localization using estrogen receptor ligand binding domain variants. In a further example, the DNA targeting domain can be linked to a first interaction component such as a first interaction domain and the KRAB domain can be linked to a second interaction component such as a second interaction domain, such that the first and second interaction domain interact.

As used herein, the term “interaction domain” means a sequence motif in a first polypeptide (e.g. first dimer interaction domain), that is capable of interacting with a binding partner comprising a sequence motif in a second polypeptide (e.g. second dimer interaction domain). In particular, the term is intended to encompass a first or second interaction dimer domain which together form a heterodimer pairs that dimerizes for example under suitable inducing conditions. Other interaction domains are specifically contemplated and can be identified by the skilled person depending on the desired characteristics. Suitable inducible interaction domain pairs include, without limitation: FKBP/FRB (FK506 binding protein/FKBP rapamycin binding), which can be induced with e.g. rapamycin or AP21967; PYL/ABI which can be induced e.g. with abscisic acid; GID1/GAI, which can be induced with e.g. gibberellin or gibberellic acid; and pMag/nMag, which can be induced by e.g. blue light and/or temperature.

The DNA targeting domain can be any suitable DNA targeting domain. Preferably, the DNA targeting domain is an enzymatically inactive sequence-specific DNA targeting protein such as a CRISPR-Cas protein, optionally dCas9, a zinc-finger DNA binding domain with custom DNA-binding specificity, tet-repressors and variants thereof, or a transcription activator-like effector (TALE) protein. Enzymatically active Cas9 can also be used when it would lead to repression, for example when the guide is a truncated guide (see for example [24]).

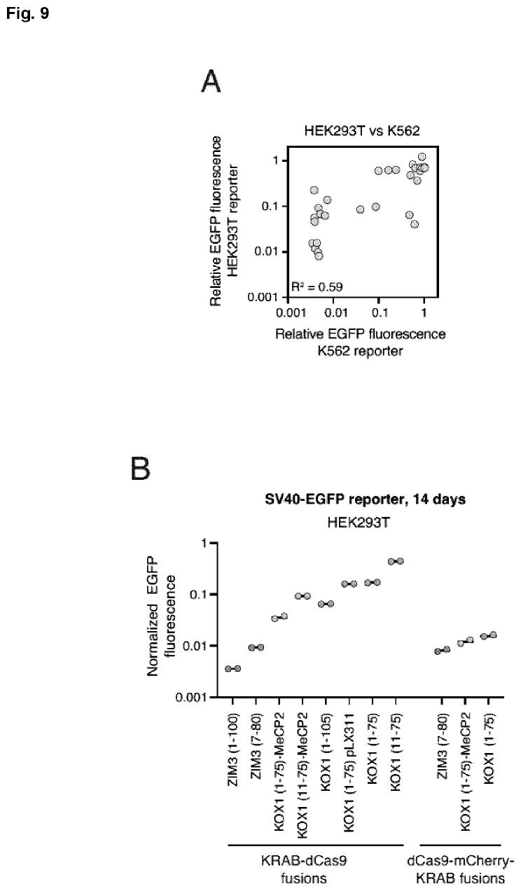

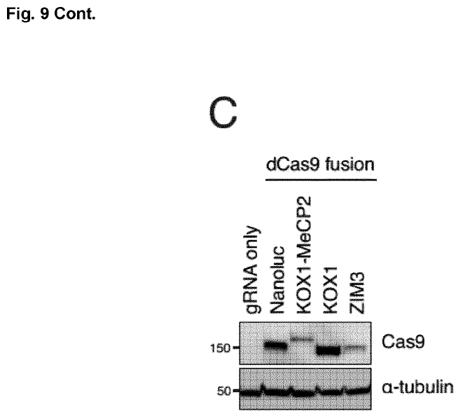

The term “KRAB domain” or Kruppel-associated box (KRAB) domain as used herein refers to a polypeptide domain of around 75 amino acids and variants thereof such as active fragments thereof or depending on the context, the nucleic acid encoding said domain, found in many Krueppel-type C2H2 zinc finger proteins (ZFPs). In the repressors described herein, the active fragment can be about 60 amino acids. For example for ZIM3 it can be VTFEDVTVNFTQGEWQRLNPEQRNLYRDVMLENYSNLVSVGQGETTKPDVILRLEQGKE PWL (SEQ ID NO: 2) which correspond to amino acids 8 to 69 (SEQ ID NO: 3) of ZIM3. For example, the active fragment can be amino acids 4-76. Examples of active fragments are disclosed herein, for example in FIG. 9. Active fragments of other KRAB domains can be identified by any suitable alignment methods, for example SMART consensus alignment.

The heterologous transcriptional repressor can be a KRAB N-terminal or a C terminal fusion, for example the order of the fusion can be KRAB domain-DNA targeting domain or DNA targeting domain-KRAB domain (see for example [25], [26], [27] and [28]). The KRAB domain can be fused to the DNA targeting domain by way of a linker. For example, glycine and glycine serine linkers can be used. Transcriptional repressors described in the Examples used a Gly₄ linker when the KRAB domain was fused to the C-terminus of dCas9 and Gly₃SerGly₃Ser when the KRAB domain was fused to the N-terminus of dCas9. Other linkers can also be used.

The terms “CRISPR-Cas” or “Cas” as used herein refer to a CRISPR Clustered Regularly Interspaced Short Palindromic Repeats-CRISPR associated (CRISPR-Cas) protein that binds RNA and is targeted to a specific DNA sequence by the RNA to which it is bound. The CRISPR-Cas is a class II monomeric Cas protein for example a type II Cas such as Cas9. The Cas9 protein may be Cas9 from Streptococcus pyogenes, Francisella novicida, A. Naesulndii, Staphylococcus aureus or Neisseria meningitidis. Optionally the Cas9 is from S. pyogenes. The Cas protein can also be Cas12a (e.g. dCas12a) for example from Acidaminococcus sp., Lachnospiraceae bacterium, or Francisella tularensis (these have been shown to work as dCas variants), Casϕ (Cas12j) and CasX (Cas12e) may also be used.

As used herein, the term “dCas9” refers to an enzymatically inactive (or dead) Cas9, which lacks DNA endonuclease activity but retains target DNA binding activity. For example, the dCAS9 comprises the sequence of CAS9 and D10A/H840A mutations in the RuvC1 and HNH nuclease domains. Optionally the dCas9 is a protein comprising an amino acid sequence with at least 80%, at least 90%, at least 95%, at least 99% or 100% sequence identity to a protein encoded by SEQ ID NO: 1 and comprising D10A/H840A mutations and retaining Cas9 target DNA binding activity (e.g. binding the gRNA and the target site). Similarly dCas12a refers to an enzymatically inactive Cas12a.

The terms “guide RNA,” “guide,” or “gRNA” as used herein refer to an engineered RNA molecule that hybridizes with a specific DNA sequence and minimally comprises a spacer sequence. The guide RNA may further comprise a protein binding segment that binds a CRISPR-Cas protein. The portion of the guide RNA that hybridizes with a specific DNA sequence is referred to herein as the nucleic acid-targeting sequence, or spacer sequence. The protein binding segment of the guide may comprise for example a tracrRNA and/or a direct repeat. The term “guide” or “guide RNA” may refer to a spacer sequence alone, or an RNA molecule comprising a spacer sequence and a protein binding segment, according to the context. The guide RNA can be represented by the corresponding DNA sequence. The guide can be a truncated guide, for example comprising 15 or fewer nucleotides of complementarity to a target site as described in [24] when the enzyme is Cas9. For example, when Cas9 interacts with a truncated guide, Cas9's DNA binding capability remains intact while its nucleolytic activity is eliminated. Any length of guide that maintains Cas binding capability can be used.

The term “spacer” or “spacer sequence” as used herein refers to the portion of the guide that forms, or is capable of forming, an RNA-DNA duplex with the target sequence or a portion thereof. The spacer sequence may be complementary or correspond to a specific CRISPR target sequence. The nucleotide sequence of the spacer sequence may determine the CRISPR target sequence and may be designed or configured to target a desired CRISPR target site.

The term “tracrRNA” as used herein refers to a “trans-encoded crRNA” which may, for example, interact with a CRISPR-Cas protein such as Cas9 and may be connected to, or form part of, a guide RNA. The tracrRNA may be a tracrRNA from for example S. pyogenes. A tracrRNA may have for example the sequence of 5′-gtttcagagctatgctggaaacagcatagcaagttgaaataaggctagtccgttatcaacttgaaaaagtggcaccgagtcggt gc-3′ (SEQ ID NO: 11). Other tracrRNAs may also be used. Suitable tracrRNAs can be identified by a person skilled in the art, including for example 5′-GTTTTAGAGCTAGAAATAGCAAGTTAAAATAAGGCTAGTCCGTTATCAACTTGAAAAAG TGGCACCGAGTCGGTGC-3′ (SEQ ID NO: 12) or 5′-GTTTCAGAGCTACAGCAGAAATGCTGTAGCAAGTTGAAAT-3′ (SEQ ID NO: 13).

The terms “CRISPR target site” or “CRISPR-Cas target site” as used herein mean a nucleic acid to which an activated CRISPR-Cas protein (e.g. a CRISPR-Cas protein such as dCas9 bound to a guide RNA) will bind under suitable conditions. A CRISPR target site comprises a protospacer-adjacent motif (PAM) and a CRISPR target sequence (i.e. corresponding to the spacer sequence of the guide to which the activated CRISPR-Cas protein is bound). The sequence and relative position of the PAM with respect to the CRISPR target sequence will depend on the type of CRISPR-Cas protein. For example, the CRISPR target site of Cas9 or dCas9 may comprise, from 5′ to 3′, a 15 to 25, 16 to 24, 17 to 23, 18 to 22, or 19 to 21 nucleotide, optionally a 20 nucleotide target sequence followed by a 3 nucleotide PAM having the sequence NGG. Accordingly, a Cas9 target site may have the sequence 5′-N₁NGG-3′ where N₁ is 15 to 25, 16 to 24, 17 to 23, 18 to 22, or 19 to 21 nucleotides in length, optionally 20 nucleotides in length or any whole number between and including 15 and 25.

The CRISPR target site can be in any suitable genomic locus. For example, the CRISPR target site can be in a promoter, enhancer, 3′UTR, or other regulatory element, in a gene, optionally an intron or exon, in a locus corresponding to a non-coding RNA, or in an intergenic region.

Target DNA located in the nucleus of a cell requires a transcriptional repressor that can enter the nucleus. Accordingly, the transcriptional repressor may be nuclear-localized and/or may comprise for example one or more nuclear localization signals (NLS), optionally one or more SV40 NLSs. Optionally the transcriptional repressor comprises two or more NLSs. Optionally the transcriptional repressor may comprise one or more N-terminal NLSs, one or more C-terminal NLSs, or one or more N-terminal and one or more C-terminal NLSs. Other configurations are specifically contemplated.

The transcriptional repressor can also be labelled with a tag. For example, suitable tags include but are not limited to Myc, FLAG, HA, V5, ALFA, T7, 6×His, VSV-G, S-tag, AviTag, StrepTag II, CBP, GFP, mCherry. For example, as described in the Examples and as shown in the SEQ ID NO: 17, the heterologous transcriptional repressor can comprise a label such as mCherry. The label can be fused at the N-terminus, the C-terminus or between two components of the heterologous transcriptional repressor such as between the DNA targeting domain and the KRAB domain.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

It must be noted that as used herein and in the appended claims, the singular forms “a”, “an”, and “the” include plural references unless the context clearly dictates otherwise.

All numerical values within the detailed description and the claims herein are modified by “about” or “approximately” the indicated value, and take into account experimental error and variations that would be expected by a person having ordinary skill in the art.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of’ or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “about” as used herein means plus or minus 10%-15%, 5-10%, or optionally about 5% of the number to which reference is being made.

It should also be understood that, in certain methods described herein that include more than one step or act, the order of the steps or acts of the method is not necessarily limited to the order in which the steps or acts of the method are recited unless the context indicates otherwise.

Although any materials and methods similar or equivalent to those described herein can also be used in the practice or testing of the present disclosure, the following materials and methods are now described.

II. Materials and Methods

Heterologous transcriptional repressors and systems for transcriptional repression using a DNA targeting domain such as an enzymatically inactive CRISPR-Cas protein and a KRAB domain are described herein. As demonstrated in the Examples, fusion proteins comprising dCas9 or dCas12a and at least one KRAB domain selected from the group consisting of ZIM3, ZNF554, ZNF264, ZNF324, ZNF354A, ZFP82, and ZNF669 and variants thereof have greater potency in transcriptional repression, interact more strongly with TRIM28, have less sensitivity to gRNA selection, and/or have less sensitivity to target location than existing KOX1 KRAB-dCas9 based transcriptional repressors. As further demonstrated in the Examples, these fusion proteins can be used for high-throughput screening, for example to conduct cell viability screens for essential genes. Also demonstrated in the Examples, an inducible transcriptional repressor comprising a dCas9-ABI1 fusion protein and a ZIM3 KRAB-PYL1 fusion protein can induce transcriptional repression in the presence of abscisic acid.

The dCas sequences can be based on sequences f

Accordingly, one aspect of the disclosure includes a heterologous transcriptional repressor comprising a DNA targeting domain, preferably an enzymatically inactive sequence specific DNA binding protein such as a CRISPR-Cas protein, and at least one KRAB domain selected from the group consisting of KRAB proteins which exhibit a stronger KRAB/TRIM28 interaction than KOX1 KRAB or KOX1 KRAB MeCP2, optionally ZIM3-KRAB, ZIM2-KRAB, ZNF554-KRAB, ZNF264-KRAB, ZNF324-KRAB, ZNF354A-KRAB, ZFP82-KRAB, and ZNF669-KRAB.

The DNA targeting domain and the KRAB domain may be covalently linked, for example as domains of a single polypeptide, or may be separate polypeptides that are linked by one or more interaction components such as interaction domains and/or interact under certain conditions. Accordingly, in one embodiment, the transcriptional repressor is a single polypeptide. In another embodiment, the transcriptional repressor further comprises a pair of (i.e. a first and a second) interaction domains, optionally dimer interaction domains, optionally a pair of inducible dimer interaction domains that dimerize under suitable conditions. For example, the transcriptional repressor may comprise a first polypeptide comprising a DNA targeting domain and a first dimer interaction domain, optionally an inducible dimerization domain, and a second polypeptide comprising a KRAB domain and a second dimer interaction domain, optionally an inducible dimerization domain, optionally the first inducible dimerization domain and second inducible dimerization domain interact in the presence of one or more inducing agents.

As shown in the Examples, the dimerization of a heterologous transcriptional repressor comprising ABM and PYL1 may be induced with the addition of abscisic acid. Accordingly, in an embodiment, the transcriptional repressor comprises a first and second inducible dimerization domain that provide for inducible transcriptional repression in the presence of an inducing agent. The skilled person can readily identify and select suitable inducible dimerization domains that may be used together. Any suitable inducible dimerization domains may be used, for example the dimerization of ABI1 and PYL1 may be induced with the addition of abscisic acid. Other inducible systems include those based on induction with rapamycin, gibberellic acid/gibberellin, and split dCas9-based systems. For example dimerization of GID1 and GAI can be induced by gibberellin, and dimerization of FKBP and FRB can be induced with rapamycin or its analogs, e.g. rapalogs. Higher-order multimerization systems, such as the SunTag system (Tenenbaum et al., 2014) are also contemplated herein.

Interaction between the DNA targeting domain and KRAB domain can also be controlled using other inducible systems. Other systems (that are not dependent on dimerization) include grazoprevir-induced stabilization (Tague et al. 2018) or tamoxifen-regulated nuclear localization using estrogen receptor ligand binding domain variants. In the case of grazoprevir-induced stabilization, the DNA targeting domain and KRAB domain would be linked by a self-cleaving NS3 protease domain. Only in the presence of grazoprevir (which inhibits NS3 activity), DNA targeting domain and KRAB domain would stay together and regulate gene expression.

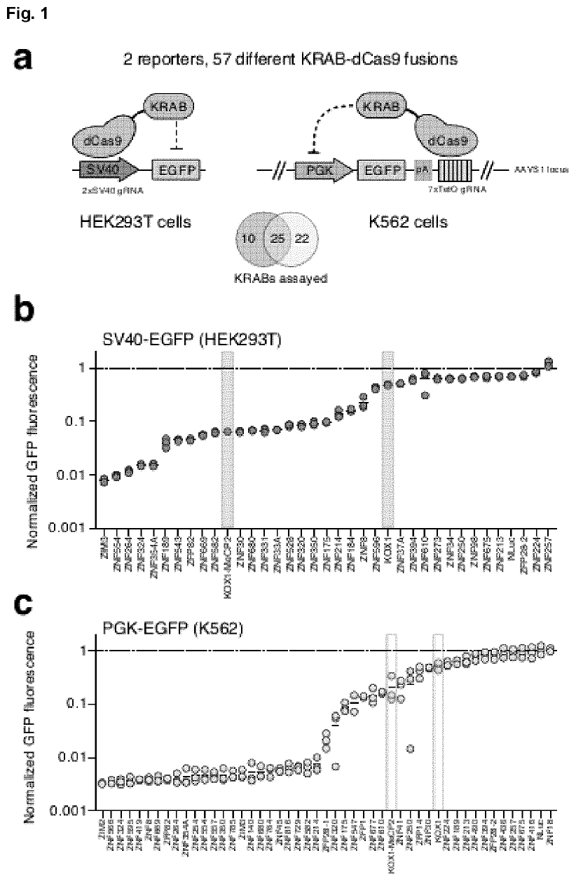

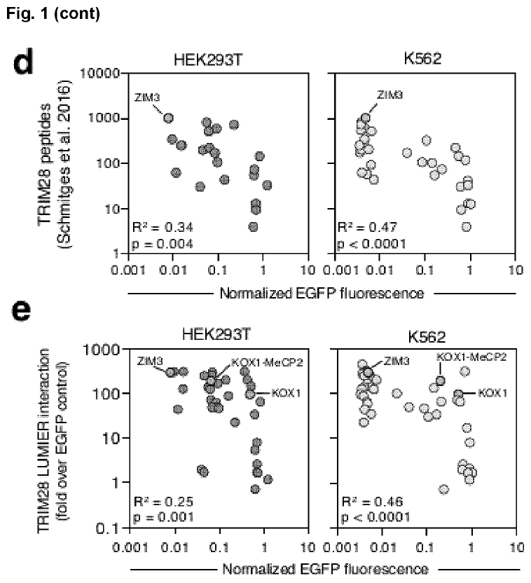

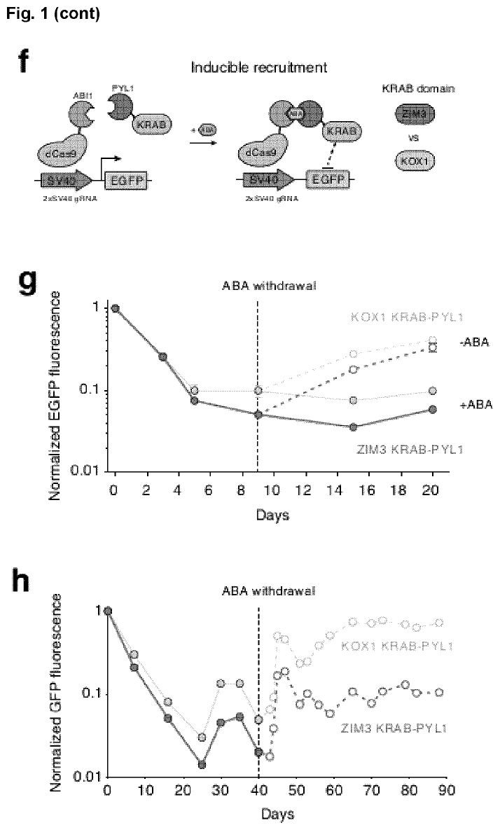

In one embodiment, the at least one KRAB domain comprises two or more KRAB domains, optionally two or more tandem KRAB domains, optionally two or more of the same or two or more different KRAB domains. The KRAB domain can for example be a KRAB domain that demonstrates greater repressor data in HEK293 and/or K562 cells as demonstrated for example in FIG. 1 b and/or c. Suitable KRAB domains include KRAB domains shown in SEQ ID Nos: 2-10 and 18 or functional variants thereof. In an embodiment the at least one KRAB domain comprises an amino acid having a sequence with at least 80%, at least 90%, at least 95%, or at least 99% sequence identity to any one of the KRAB domains in SEQ ID NO: 2-10 and 18, for example the KRAB domain related to accession number Q96PE6-1 (UniProt), and which retains (e.g. is as effective at) transcriptional repression activity and/or interaction with TRIM28 as for example said KRAB domain (e.g SEQ ID NO: 2-10 or 18 or for example the KRAB domain related to accession number Q96PE6-1) or for example KOX1 KRAB MeCP2. In an embodiment the at least one KRAB domain is a ZIM3 KRAB domain, optionally having amino acid having a sequence with at least 80%, at least 90%, at least 95%, or at least 99% sequence identity to accession numbers Q96PE6-1 (UniProt) or SEQ ID NO: 2 (or the KRAB domain of SEQ ID NO: 3) and which is as effective at (e.g. retains) transcriptional repression activity and/or interaction with TRIM28 as for example said KRAB domain (e.g. ZIM3 KRAB domain) or for example KOX1 KRAB MeCP2.

As effective as used herein means retains at least 80%, at least 85%, at least 90%, at least 95% or at least 99% transcriptional repression activity and/or co-repressor interaction compared to the wild-type KRAB domain (i.e. non-variant KRAB). Transcriptional repressor activity and/or co-repressor interaction of variants such as truncations can be determined for example using the methods described herein. For example transcriptional repressor activity can be determined using the EGFP reporter system(s) described in the Examples. Variants can be tethered to the same reporter or endogenous context while controlling for expression levels of each DNA-binding moiety (e.g. dCas9). Any differences detected in induced expression of the reporter or target genes when compared to the parental KRAB can be contributed to the effect of the variant. Co-repressor interaction can be determined for example by affinity purification-mass spectrometry (AP-MS) e.g. as shown in the Examples.

The KRAB domain of ZIM3 is 62 aa (aa 8-69) of ZIM3. Some KRAB domains are longer.

In one embodiment, the at least one KRAB domain is a human KRAB domain. In another embodiment, the KRAB domain comprises at least 55, at least 60, at least 65 or at least 70 amino acids. In another embodiment, the KRAB domain comprises one or more mutations.

In one embodiment, the at least one KRAB domain is two or three KRAB domains, optionally in tandem.

The DNA targeting domain can be selected from a variety of DNA targeting domains. For example, the DNA targeting domain can be selected from an engineered or natural zinc finger DNA binding domain, transcriptional activator-like effector (TALE), dCas9, dCas12 or other Cas-family proteins, or other natural DNA-binding domains (DBDs) from eukaryotes or prokaryotes (e.g. Forkhead, basic helix-loop-helix, leucine zipper, homeodomain, nuclear hormone receptor). In the case of custom ZFs, TALEs or Cas family proteins, KRAB domains could be brought to a single locus in the genome in a controlled manner. In the case of natural DNA-binding domains, KRAB domains would be brought to all loci that a given transcription factor binds to, thereby augmenting/replacing the function of the endogenous TF.

In an embodiment, the enzymatically inactive sequence specific DNA binding protein is a CRISPR-Cas protein such as dCas9. Enzymatically inactive CRISPR-Cas proteins which retain gRNA and target DNA binding activity. For example, mutation of D10A/H840A introduces mutations in the RuvC1 and HNH nuclease domains and results in inactivation. In an embodiment, the CRISPR-Cas protein is dCas9 having an amino acid sequence of SEQ ID NO: 1 or an amino acid sequence with at least 80%, at least 90%, at least 95%, or at least 99% sequence identity to SEQ ID NO: 1 and comprises D10A/H840A or corresponding mutations and which retains gRNA and target DNA binding activity. Other enzymatically inactive CRISPR-Cas proteins falling within the scope of the disclosure can be identified by the skilled person.

Exemplary heterologous transcriptional repressor nucleic acids are provided in SEQ ID NO: 14, 16 and 17. In an embodiment, the heterologous transcriptional repressor may comprise an amino acid sequence encoded by said nucleic acids, or an amino acid sequence with at least 80%, at least 90%, at least 95%, or at least 99% sequence identity to an amino acid sequence encoded by the DNA targeting domain and KRAB domain portions of and SEQ ID NO: 14, 16 or 17. The activity of the encoded polypeptides (fusion or when expressed and activated) of such polypeptides is as effective (e.g. provides as effective transcriptional repression) as for example SEQ ID NO: 14, 16 or 17.

In an embodiment, the transcriptional repressor comprises a first and second interaction component (e.g. inducible protein dimerization domain) that provide for inducible transcriptional repression in the presence of an inducing agent. The skilled person can readily identify and select suitable interaction components such as inducible dimerization domains that may be used together. Any suitable inducible combination of protein dimerization domains and inducing agents may be used, for example the dimerization of ABI1 and PYL1 may be induced with the addition of abscisic acid. Other inducible systems include those based on induction with rapamycin, gibberellic acid/gibberellin, and split dCas9-based systems. For example dimerization of GID1 and GAI can be induced by gibberellin, and dimerization of FKBP and FRB can be induced with rapamycin or its analogs, e.g. rapalogs. Other inducing agents can include auxin for inducing auxin-based dimerization, where auxin treatment leads to interaction of TIR1 leucine-rich repeat region (LRR) with auxin-inducible degron (AID) sequence or tamoxifen and related molecules estrogen for receptor ligand-binding domain (LBD) fusions, where ER LBD keeps the construct in the cytoplasm until treatment with tamoxifen.

In an embodiment, the KRAB domain is fused to the DNA targeting domain by way of a linker. In an embodiment, two or more KRAB domains are fused together by way of one or more linkers. For example, glycine and glycine serine linkers can be used. Transcriptional repressors described in the Examples used a Gly₄ linker when the KRAB domain was fused to the C-terminus of dCas9 and Gly₃SerGly₃Ser when the KRAB domain was fused to the N-terminus of dCas9. Other linkers can also be used.

In an embodiment, the transcriptional repressor comprises one or more nuclear localization signals (NLS). Any suitable NLS can be used. Optionally the NLS is an SV40 NLS. The one or more NLS can be one or more N-terminal NLS, one or more C-terminal NLS, one or more internal NLS, and/or combinations thereof.

As described herein, the transcriptional repressor may be encoded by a nucleic acid and/or expressed from an expression construct. Accordingly, one aspect of the disclosure is a nucleic acid encoding a transcriptional repressor described herein. For example, the nucleic acid may be a nucleic acid of any one of SEQ ID Nos: 14, 16 or 17, a sequence with at least 80%, at least 90%, at least 95%, at least 99% or 100% sequence identity to SEQ ID NO: 14, 16 or 17, wherein the heterologous transcriptional repressor, for example repressors transcription about as effectively as SEQ ID NO: 14, 16 or 17, for example at least 80% as effectively, at least 85% as effectively, or at least 90% as effectively, for example as assessed in assay as described herein. The sequence identity is for example relative to the DNA targeting domain and the KRAB domain. Other portions, linkers, NLS etc can be completely different.

A related aspect is an expression construct comprising the nucleic acid encoding the transcriptional repressor operably linked to a promoter and a transcription termination site. Any suitable promoter may be used. Suitable promoters can be identified by a person skilled in the art, and may include for example CMV, EF1A, or PGK. For example, the promoter and enhancer sequence of SEQ ID NO: 15 can be used in an expression construct. Inducible promoters may also be used.

In one embodiment, the construct is a vector. Any suitable vector may be used. Suitable vectors can be identified by a person skilled in the art, and may include a viral vector, optionally a lentiviral vector or an adenoviral vector. The vector may also be a self-replicating viral RNA replicon or plasmid.

Suitable vectors may comprise for example a promoter for expressing a nucleic acid of interest (e.g. gRNA or repressor or a component thereof), polyA tail, 3′UTR elements like WPRE to increase stability of expression, insulator sequences, lentiviral packaging signals, and/or an antibiotic resistance marker. The components such as promoter, be a eukaryotic promoter. A nucleic acid can be operatively linked to a promoter sequence, optionally a eukaryotic promoter sequence, and other elements. Additional suitable components can be identified by a person skilled in the art.

In another embodiment, the transcriptional repressor, nucleic acid, construct, or vector is in a cell. Any suitable cell may be used and can be determined by the skilled person on the basis of the desired application. The cell may be from any organism, optionally a mammal. Optionally the cell is a mammalian cell such as a human cell or a rodent cell, optionally a mouse cell. Optionally the cell is a cell line. The cell line may be any suitable cell line. The cell can be a primary cell. In an embodiment, the cell is a T cell. In another embodiment, the cell is a disease cell, optionally a cancer cell. In yet another embodiment, the cell is a stem cell, optionally an induced pluripotent stem cell.

The transcriptional repressor, nucleic acid, construct, or vector may be introduced into the cell in any suitable manner, for example by transfection. Suitable transfection reagents and methods are routinely practiced in the art and can be identified by the skilled person. Optionally, the construct is a viral vector, optionally a lentiviral vector, and is introduced into the cell by transduction. Suitable transduction methods are routinely practiced in the art and can be identified by the skilled person.

In some embodiments the cell is stably expressing the heterologous transcriptional repressor, optionally the cell is stably transduced, for example prepared using a virus comprising a nucleic acid encoding the heterologous transcriptional repressor.

Another aspect is a transcriptional repression system comprising the transcriptional repressor described herein, a nucleic acid encoding the transcriptional repressor, or construct or vector comprising said nucleic acid or a cell expressing the transcriptional repressor. In the case of a system based on CRISPR-Cas, the system comprises at least one gRNA. In the case of a system based on inducible dimerization domains, the system optionally comprises at least one inducing agent.

Also provided is a composition comprising a heterologous transcriptional repressor described herein, a nucleic acid described herein, a construct described herein, a vector described herein, a cell described herein and/or a transcriptional repression system described herein. The composition can comprise a carrier, such as BSA, or a diluent suitable according to the composition components, optionally water or buffered saline. The composition can comprise multiple components such as transcriptional repressors, nucleic acids, constructs, vectors or cells comprising the same or different elements.

Also provided herein is a kit for example for repressing transcription of a target gene or performing a method described herein, the kit comprising a transcriptional repressor described herein, a nucleic acid, expression construct, or vector encoding a transcriptional repressor described herein, or a cell expressing the transcriptional repressor described herein, and optionally a vial housing the transcriptional repressor, nucleic acid, expression construct, vector, cell or composition. The kit can comprise multiple of one or more of the aforementioned components. Optionally the kit comprises a gRNA expression construct (e.g. a nucleic acid encoding the gRNA operatively linked to a promoter sequence, optionally a eukaryotic promoter sequence), an inducing agent, and/or instructions for carrying out the methods described herein.

Also described herein are methods of repressing transcription of a target gene in a cell. As demonstrated in the Examples, a transcriptional repressor of the disclosure can be targeted to a genomic locus such as a promoter to repress transcription of a target gene in a cell.

As described herein another level of control for transcriptional regulation can be added with chemically induced dimerization with e.g. rapalogs or abscisic acid. In this case, one half (e.g. a DNA-binding moiety) would be fused to FKBP or PYL1, and the other half (e.g. the KRAB domain) fused to FRB or ABM. Treatment with rapalog or abscisic acid would induce the interaction between FKBP and FRB or PYL1 and ABI1, respectively, leading to temporally regulated gene expression. As shown in the Examples, the dimerization of a heterologous transcriptional repressor comprising ABI1 and PYL1 may be induced with the addition of abscisic acid. The skilled person can readily identify and select suitable inducible dimerization domains and inducing agents that may be used together. Any suitable inducible combination of protein dimerization domains and inducing agents may be used, for example the dimerization of ABI1 and PYL1 may be induced with the addition of abscisic acid. Other inducible systems include those based on induction with rapamycin, gibberellic acid/gibberellin, and split dCas9-based systems. For example dimerization of GID1 and GAI can be induced by gibberellin, and dimerization of FKBP and FRB can be induced with rapamycin or its analogs, e.g. rapalogs. Higher-order multimerization systems, such as the SunTag system (Tenenbaum et al., 2014) are also contemplated herein.

Interaction between the DNA targeting domain and KRAB domain can also be controlled using other inducible systems. Other systems (that are not dependent on dimerization) include grazoprevir-induced stabilization (Tague et al. 2018) or tamoxifen-regulated nuclear localization using estrogen receptor ligand binding domain variants. In the case of grazoprevir-induced stabilization, the DNA targeting domain and KRAB domain would be linked by a self-cleaving NS3 protease domain. Only in the presence of grazoprevir (which inhibits NS3 activity), DNA binding domain and KRAB domain would stay together and regulate gene expression.

Accordingly, one aspect of the disclosure is a method of repressing expression of a target gene in a cell, the method comprising introducing into the cell a transcriptional repressor described herein, and culturing the cell under suitable conditions such that the DNA targeting domain guides the transcriptional repressor to the target site and the at least one KRAB domain represses transcription of the target gene. In an embodiment, where the transcriptional repressor comprises CRISPR-Cas, the method further comprises introducing into the cell at least one gRNA that targets a desired genomic locus in the cell, and culturing the cell under suitable conditions such that the at least one gRNA associates with the CRISPR-Cas protein and guides the CRISPR-Cas protein to guide the transcriptional repressor to a CRISPR target site such that the at least one KRAB domain represses transcription of the target gene. In an embodiment where the transcriptional repressor comprises an inducible dimerization domain in each of the DNA targeting domain and in the KRAB domain, the method further comprises introducing into the cell at least one inducing agent and culturing the cell under suitable conditions that the first and second inducible dimerization domains associate such that the at least one KRAB domain represses transcription of the target gene. In an embodiment, the cell is in a subject. Accordingly in an embodiment, the method is for repressing expression of a target gene in an animal model For example the present methods can be used in mouse or other rodent models and in mammals such as humans, in ex vivo or in vivo applications. For example the systems can be used in CAR-T circuits or controlling gene expression after AAV- or lipid nanoparticle based delivery.

The methods described herein can be used to identify or screen for one or more genomic loci that are important for cell viability or a phenotype of interest. By way of example, the methods described herein can be used to screen for genes or regulatory elements thereof that are important for resistance or sensitivity to a toxin of interest such as diphtheria toxin. In another example, the methods described herein can be used to identify regulatory elements that are important for expression of a protein of interest such as CD81. In a further example, the methods described herein can be used to in high-throughput screening methods to essential or non-essential genes in a cell type by screening for gRNAs that are over- or under-represented in a cell population under certain conditions e.g. drug treatment over time. They can be used to identify regulatory elements that respond (or do not respond) to KRAB domain based repression, or to identify regulatory elements essential for cancer cell proliferation. Other applications can be determined by a person skilled in the art.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### III. Examples

Example 1. Identification of highly potent KRAB domains.

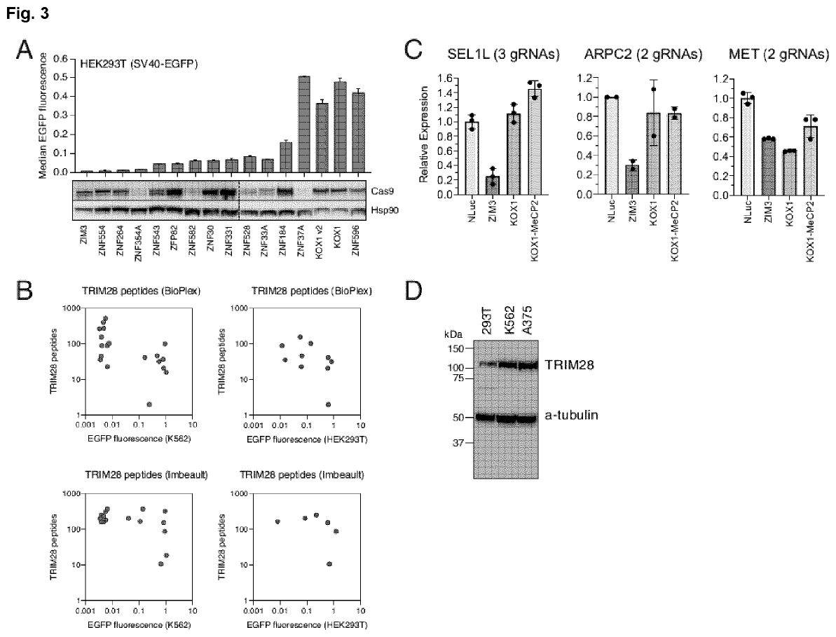

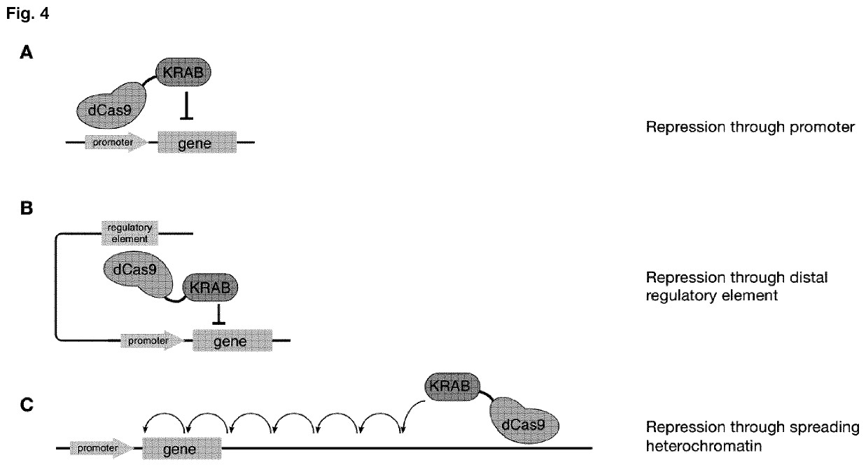

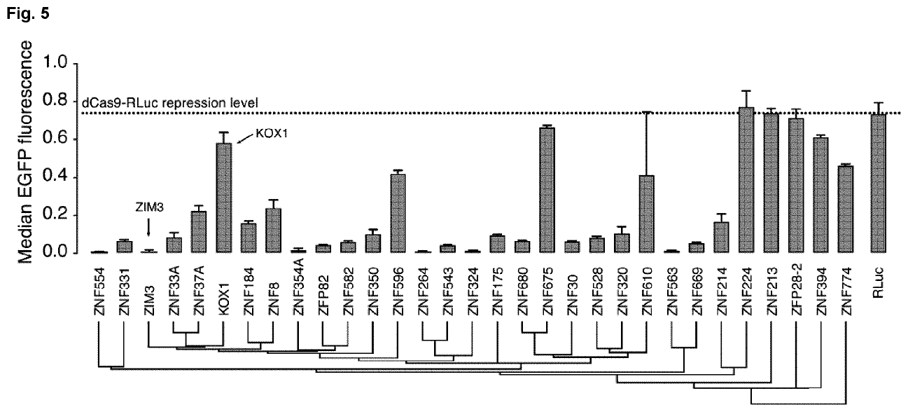

The human genome encodes over 350 KRAB domain proteins [11-13]. It was postulated that KRAB domains would differ in their ability to silence gene expression when fused to dCas9. 57 human KRAB domains were individually fused to the N terminus of dCas9 and assayed for activity when recruited to one of two different genomically integrated reporter constructs. In one, they were recruited to a SV40 promoter driving EGFP expression in HEK293T cells [14], and in the other tethered to a 7×TetO array downstream of the polyadenylation site of a PGK1-EGFP-pA reporter in K562 cells [15] (FIG. 1a). KRAB domains had strikingly different effects, spanning from almost complete silencing to no change at all (FIG. 1b,c and FIG. 5). The differences were not explained by expression levels of the dCas9 fusion (FIG. 3). Moreover, the results were consistent between the two reporters (Rsq=0.59; FIG. 1A), indicating that the effects were not cell-type specific (see FIG. 9). KRAB was also fused to the C-terminus of dCas9 with similar results (see Example 5 and FIG. 3).

Interestingly, the KRAB domain from KOX1 was not a particularly strong repressor compared to several other KRAB domains, such as that from ZIM3 gene (FIG. 1b,c and FIG. 5). Even KOX1 KRAB-MeCP2 fusion, which was more potent than KOX1 KRAB only (FIG. 1b,c and FIG. 5), failed to fully silence the reporters. It was then determined that the results were not due to vector design by assaying a previously reported lentiviral KOX1-dCas9 construct [9,16] (FIG. 1B and FIG. 9). The modest improvement in the potency of the previously reported construct was likely due to the presence of additional nine amino acids N-terminal to the annotated KRAB domain. Adding flanking sequences to KOX1 KRAB domain in the vector design slightly improved its activity—but it remained clearly weaker than many other KRAB domains.

KRAB domains induce silencing by interacting with TRIM28/KAP1, which acts as a scaffold for a heterochromatin-inducing protein complex [17]. However, not all KRAB domain proteins interact with TRIM28 [10,15]. To assess the relationship between TRIM28 binding and silencing, KRAB activity was compared to the number of TRIM28 peptides recovered in three independent affinity-purification/mass spectrometry datasets with full-length KRAB-domain proteins [10,15,16]. In each case, there was a significant positive correlation between the strength of KRAB/TRIM28 interaction and the extent of repression in the reporter assay (FIG. 1d and FIG. 3). The interaction of 49 KRAB-EGFP fusions with TRIM28 was then profiled using LUMIER, a pairwise quantitative interaction assay [14]. Again, there was a significant positive correlation (Rsq=0.46 and 0.25 for K562 and HEK293T, respectively; FIG. 1e). Thus, the interaction strength with TRIM28 appears to be a major determinant of the silencing activity of KRAB domains. Interestingly, the KOX1 KRAB-MeCP2 fusion interacted more strongly with TRIM28 than KOX1 KRAB only (FIG. 1e), suggesting that the improved potency of KRAB-MeCP2 fusions in CRISPRi could due to enhanced TRIM28 interaction.

### Methods are as Described in Example 4

### Example 2—Characterization of Temporal Dynamics

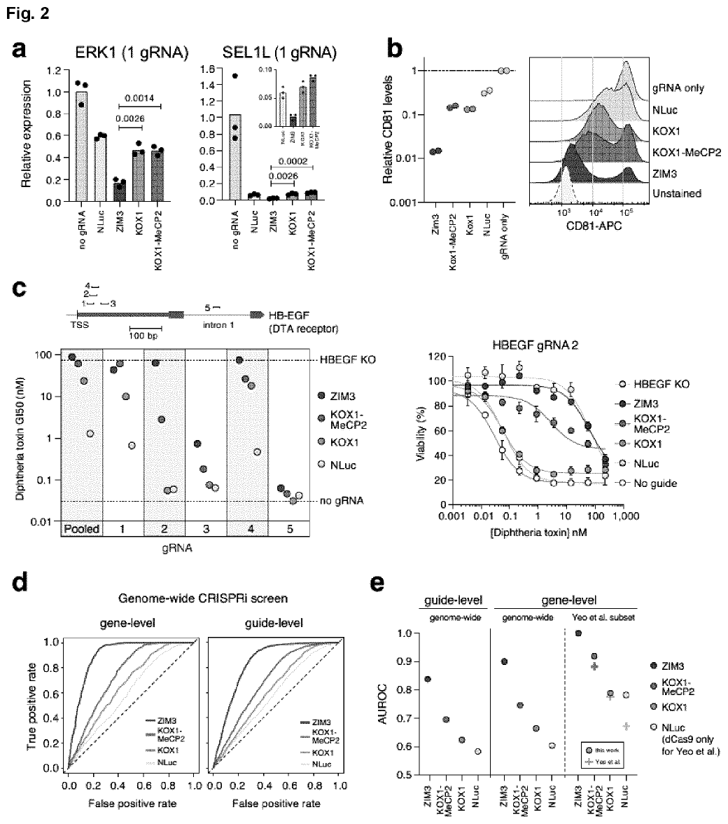

The difference in KRAB-domain activity might be explained either by their intrinsic potency or by their distinct temporal dynamics. To differentiate between these options, a chemically induced dimerization system was used. The interaction between plant proteins ABM and PYL1 is induced by abscisic acid (ABA) in a reversible manner (FIG. 1f). A clonal SV40-EGFP reporter cell line expressing ABI1—dCas9 and a gRNA targeting two sites on the SV40 promoter was generated. These cells were then infected with either ZIM3 or KOX1 KRAB domain fused to PYL1 and treated with ABA for 20 d (FIG. 1g, solid lines). Both ZIM3 and KOX1 KRAB domains induced repression with similar dynamics, but ZIM3 KRAB reached higher level of repression despite lower expression level (FIG. 1g, FIG. 2c, and FIG. 9). When ABA was withdrawn after 9d, derepression occurred with similar dynamics with both KRAB domains (FIG. 1g, dashed lines). Thus, the difference in KRAB-domain potency is not due to slower dynamics of KOX1 KRAB induced repression. However, after 40-d silencing followed by ABA washout, KOX1 KRAB-PYL1 expressing cells restored EGFP expression almost fully, whereas in the ZIM3 KRAB-PYL1 expressing cells EGFP expression reached only 10% of the original levels (FIG. 1h). These results suggest that prolonged recruitment of ZIM3 KRAB can induce permanent silencing of expression, whereas KOX1 KRAB appears to function mainly through reversible mechanisms. This is consistent with previous studies showing that KRAB domains can mediate silencing via both reversible and irreversible mechanisms.

### Example 3. Benchmarking ZIM3 KRAB-dCas9 Fusion in CRISPRi Applications

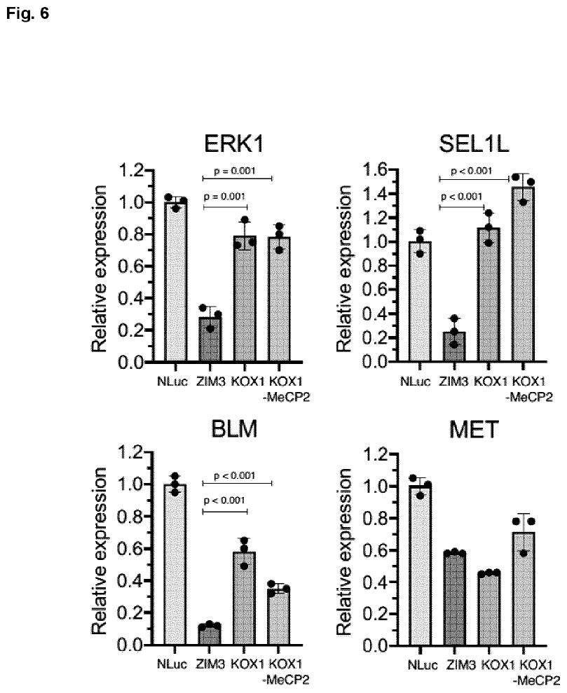

A highly potent KRAB domain was tested for the ability to outperform current versions of CRISPRi. The ZIM3 KRAB domain was the strongest repressor tested in both cell lines and among the strongest TRIM28 interactors. First, ZIM3-KRAB-dCas9, KOX1-KRAB-dCas9, KOX1-KRAB-MeCP2-dCas9 and negative control Nanoluc-dCas9 were recruited to five endogenous promoters in HEK293T cells and silencing was assessed by qRT-PCR. In four out of five cases, ZIM3 KRAB silenced expression significantly better than KOX1 KRAB or KOX1 KRAB-MeCP2 (FIG. 2A, FIG. 3, and FIG. 6). To test whether these differences translate to protein levels, these constructs were targeted to the promoter of CD81, a cell surface antigen. ZIM3 KRAB was able to reduce CD81 surface protein expression up to 10-fold better than the other two constructs (FIG. 2B).

The effect of KRAB constructs on counteracting the toxicity of Diphtheria toxin (DTA) was assayed. DTA toxicity is entirely dependent on HB-EGF, the cell-surface receptor for the toxin [18]. As shown, silencing of HBEGF, the receptor for Diphtheria toxin (DTA), with dCas9-ZIM3 leads to a significantly higher resistance to DTA than repression by KOX1 KRAB or KOX1-MeCP2. Notably, even small quantities of the receptor are sufficient for toxin endocytosis, leading to cell death. The constructs were first targeted to the HB-EGF promoter with a pool of five gRNAs. In this case, all KRAB constructs rendered the cells significantly resistant to DTA, although the effect of ZIM3 KRAB was the closest to that of HB-EGF knockout cells (FIG. 2C). However, the differences were more pronounced when each gRNA was tested separately. With the exception of one completely inactive gRNA, ZIM3 KRAB was the most potent effector. With three gRNAs, its effect was almost indistinguishable from HB-EGF KO cells (FIG. 2C). Furthermore, ZIM3 KRAB appeared less sensitive to gRNA selection than the other two constructs. In particular, HB-EGF gRNA #2 showed no activity with KOX1 KRAB, was modestly improved with the addition of MeCP2, and was fully effective with ZIM3 KRAB (FIG. 2C).

The sensitivity of the KRAB constructs to gRNA location was also tested. ZIM3 KRAB and KOX1 KRAB-MeCP2 were targeted upstream and downstream of a GFP reporter in mouse embryonic fibroblasts. Both constructs partially silenced GFP when targeted to the promoter, with KOX1 KRAB-MeCP2 being slightly more efficient than ZIM3. However, when targeted downstream of the reporter, only ZIM3 silenced GFP expression, demonstrating that ZIM3 can silence gene expression more effectively from a distal site than KOX1 KRAB-MeCP2.

Finally, the performance of the three KRAB effector constructs was investigated in a large-scale unbiased screen. A cell viability dropout screen was performed in HEK293T cells stably expressing ZIM3 KRAB-dCas9, KOX1 KRAB-dCas9, KOX1 KRAB-MeCP2-dCas9, or Nanoluc-dCas9. These cells were infected with the recently described Dolcetto Set A gRNA library targeting 18,901 genes with three gRNAs per gene and measured gRNA depletion after 21 days. The relative depletion of gRNAs targeting gold-standard essential genes and non-essential genes with each construct was compared with receiver operating characteristics (ROC) curves. As reported previously, KOX1 KRAB-MeCP2 was more efficient than KOX1 KRAB alone based on area under the ROC (AUROC), whether calculated at the gRNA level or gene-level (gRNA-level 0.62 vs 0.70, gene-level 0.66 vs 0.75; FIG. 2D, E). In contrast, ZIM3 KRAB significantly outperformed both constructs with AUROCs of 0.84 (gRNA-level) and 0.90 (gene-level; FIG. 2D, E). It should be noted that the AUROC values reported here are somewhat lower than what has been reported for CRISPRi screens in other cell lines like K562 and A3759 but in line with the previously conducted screen in HEK293T cells [8] (FIG. 2D, E). The lower efficiency may be due to the lower expression level of TRIM28 in HEK293T cells (FIG. 3). Nevertheless, these results strongly support the initial results with reporter genes and individual endogenous genes: ZIM3 KRAB domain is an exceptionally strong transcriptional repressor.

Taken together, highly potent KRAB domains that are significantly more effective in target gene silencing and less sensitive to gRNA selection than currently existing systems are described herein. The ZIM3 KRAB repressor may be particularly valuable in applications that require highly robust gene silencing or that are limited by the requirement for multiple gRNAs targeting each gene, such as genetic interaction profiling or Perturb-Seq [19-21].

### Example 4

### Methods

Cell culture. All HEK293T cells, including the SV40-EGFP reporter cell line (gift from Lei Stanley Qi lab, Stanford University), were maintained in Dulbecco's Modified Eagle's Medium (DMEM) with 10% fetal bovine serum (FBS), and 1% penicillin-streptomycin. Since the SV40-EGFP reporter cell line was originally generated with a random integration of lentivirus, a clonal line was made to ensure homogeneously high expression levels of EGFP and its targeting gRNA. K562 reporter cells (a gift from Angelo Lombardo lab, IRCCS San Raffaele Scientific Institute) were maintained in Iscove's Modified Dulbecco's (IMDM) supplemented with 10% FBS and 1% penicillin-streptomycin. K562 cells were infected with a single gRNA targeting the TetO array downstream of an EGFP-expression cassette integrated in the AAVS1 locus. These cells were then used for subsequent silencing experiments while always selecting for the same high EBFP2+ cells, used as a surrogate for gRNA expression. These cells were then transduced at high multiplicity of infection with lentivirus containing each repressor variant fused to dCas9. HEK293T and K562 reporter cells were treated for two rounds of selection with 6 and 10 μg/ml blasticidin respectively and maintained for three weeks before measurement of reporter with flow cytometry. All cell lines were routinely tested for mycoplasma contamination.

Plasmid design. Individual repressors were cloned into a Gateway-compatible lentiviral vector with a C-terminal human codon-optimized Streptococcus pyogenes dCas9 with an N-terminal SV40 nuclear localization signal (NLS) and two C-terminal SV40 NLSs. The HEK293T cell line constitutively expressing EGFP was initially targeted as explained previously. Western blots probing for Cas9 were performed on a subset of HEK293T stable cell lines to account for any variable levels of expression. When testing the K562 reporter line, a C-terminal P2A-dsRed was added to dCas9 as a surrogate for expression and only high dsRed+ cells were gated during subsequent measurements with flow cytometry. Each KRAB domain was either PCR amplified or synthesized directly, allowing for 30 additional amino acids flanking the KRAB domain, taken from the endogenous protein and as annotated by UniProt whenever possible. The sgRNAs targeting individual endogenous genes were cloned into a U6-based puromycin-resistant pLCKO. The sgRNAs targeting the reporters were cloned into a modified pLCKO to co-express EBFP2 from an hPGK promoter.

Virus production. For small scale virus production, lentivirus was generated by transiently transfecting low passaged HEK293T cells on 6-well plates with the construct of interest, psPAX2, and pVSV-G at a ratio of 8:6:1. Transfection was performed using Lipofectamine 2000 (Thermo) according to the manufacturer's protocol. For large scale production of the pooled gRNA library, HEK293T cells were transfected on multiple 15-cm dishes using XtremeGENE 9 (Roche) as previously described [29]. 6-8 hours post transfection, media was changed to harvest media (DMEM+1.1 g/100 mL BSA) and virus was collected 36 hours post transfection by passing through a 0.45 μm filter.

RT-qPCR. HEK293T cells stably expressing repressor-dCas9 fusions were independently infected as technical replicates with each individual or pool of gRNAs on 48-well plates. 24 hrs post infection, cells were selected with 1 μg/ml puromycin and passaged on 24-well plates for 9 days. Total RNA was extracted using TRI reagent (Sigma). Luna universal one-step RT-qPCR kit (NEB) was used on 50 ng of total RNA with cycling conditions: 55° C. for 10 min, 95° C. for 1 min, 40 cycles of 95° C. for 10 sec and 60° C. for 30 sec (plate read), followed by a 60-95° C. melt curve. Primers were designed to span exon-exon junctions and expression was normalized to the housekeeping gene RPL13A via the 2—ΔΔCt method.

Sensitivity to Diphtheria toxin. HEK293T cells stably expressing repressor-dCas9 fusions were transduced with each individual or equimolar pool of gRNAs on 6-well plates. 24 hrs post infection, cells were passaged onto a 10-cm dish and selected with 1.5 μg/ml puromycin for 3 days. After selection was complete, cells were seeded on 96-well plates 24 hours prior to toxin treatment at to be at 40% confluency the next day. Diphtheria toxin was serially diluted in a storage buffer right before application. Seeded cells were treated with serially diluted Diphtheria toxin for 48 hours. Toxin containing media were removed, cells were washed once with 1×PBS and were further incubated with fresh media containing alamarBlue reagent at 1:5 ratio for 90 and 180 minutes. The cell viability was recorded by measuring alamarBlue dye fluorescence using a plate reader (Biotech). HB-EGF knockout cell line was generated by transfecting px459 plasmid with a gRNA targeting HBEGF from TKOv3 library. Transfected cells were selected with 1.5 ug/ml puromycin for 3 days. The knock-out was confirmed by the surveyor assay.

CRISPRi. Viral titer for each cell line was determined on 15-cm dishes with a range of virus volumes (0, 50, 100, 150, 200, 250 and 500 μl). Validated HEK293T cells with heterogeneous expression of each repressor-dCas9 derivative was transduced at a dose resulting in ˜30% viability. Selection was done with 1 μg/ml puromycin 24 hrs post transduction resulting in complete selection within 48-72 hrs (TO). At ˜30% infection efficiency, enough cells were transduced to achieve an initial representation of >500-fold for each dCas9 variant. Cells were passaged and maintained in two technical replicates while maintaining 250-fold coverage throughout. Cells were pelleted after selection T14 and T21 and flash frozen on dry ice for genomic DNA isolation.

LUMIER. 293T cells stably expressing NLuc-tagged TRIM28 at the C-terminus were transfected with KRAB domains C-terminally tagged with EGFP-3×FLAG using polyethylenimine. Two days post transfection, cells were washed in PBS, lysed in HENG buffer and transferred into 384-well plates coated with monoclonal anti-FLAG M2 antibody. Plates were incubated in the cold for 3 hrs, washed with HENG buffer and luminescence was measured with a plate reader. Afterwards, ELISA signal against HRP-conjugated anti-FLAG antibody was measured as a control for expression.

### Example 5

Next, we tested whether difference seen in potency between ZIM3 and KOX1 was due to orientation of fusion proteins. We modified the original CRISPRi plasmid (27) by inserting an mCherry fused either to KOX1-KRAB, KOX1-MeCP2 or ZIM3-KRAB at the C-terminus of dCas9. SV40-EGFP cells expressing a single gRNA targeting the promoter were transduced with each repressor. Cells expressing similar levels of mCherry, surrogate for dCas9-KRAB, were gated two weeks post infection. ZIM3 KRAB was more potent than KOX1 KRAB or KOX1 KRAB-MeCP2 fusion even when fused to the C terminus of dCas9 in a widely used backbone. Results are shown in FIG. 3. Additional methods are as described in Example 4.

### Example 6

### Inducible Systems

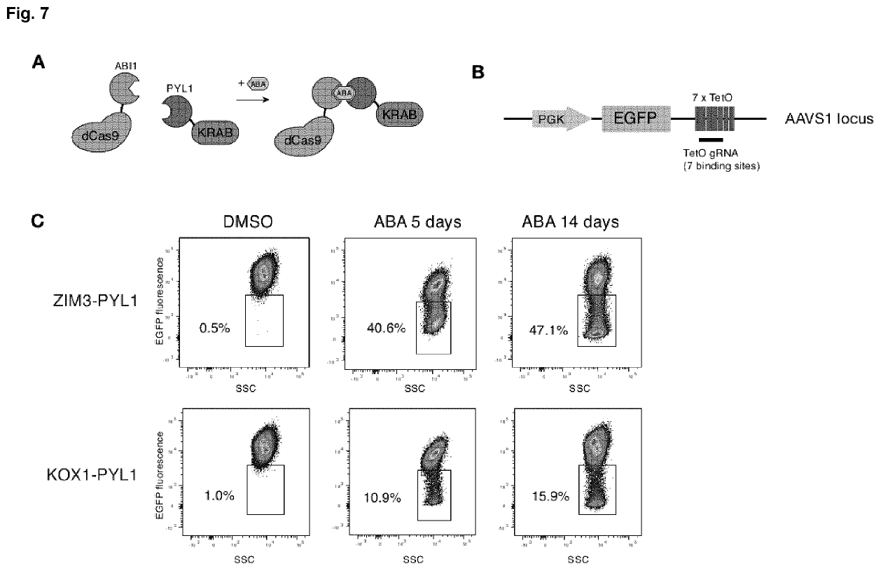

The performance of the ZIM3 KRAB repressor was tested in an inducible repression system as described in [14]. K562 reporter cells were transduced with ABI-dCas9 and a single gRNA targeting the TetO array downstream of an EGFP expression cassette integrated in the AAVS1 locus. These cells were then infected at high multiplicity of infection with lentivirus containing either ZIM3 or KOX1 KRAB domains fused to PYL1. After two rounds of selection, cells were treated with 100 μM of abscisic acid and EGFP levels after 5 and 14 days of recruitment were measured by flow cytometry (FIG. 7). Similar to previous experiments using direct fusions, ZIM3-PYL1 showed superior silencing compared to KOX1-PYL1. Additional methods are as described in Example 4.

### Example 7—Assessing Off-Target Effects KRAB-Mediated Silencing

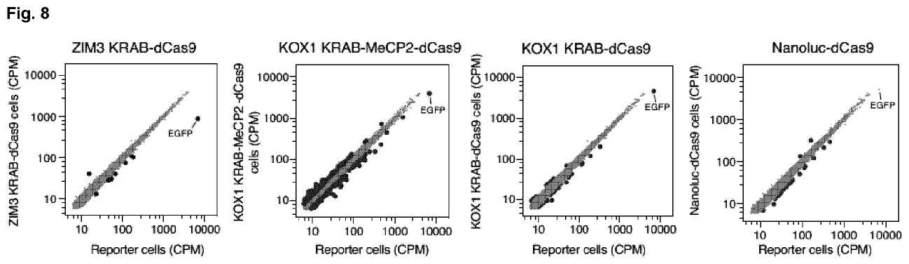

More potent repression of on-target genes could lead to more pronounced effects on potential off-targets. This was assessed by sequencing the transcriptome of the SV40-EGFP reporter cells 30 days after infection with dCas9 fused to ZIM3 KRAB, KOX1 KRAB, KOX1 KRAB-MeCP2 or Nanoluc. In ZIM3 KRAB-dCas9 infected cells, ten genes in addition to EGFP itself were significantly down- or upregulated (FIG. 8). This was, in fact, fewer affected genes than for any other construct (FIG. 8). Moreover, none of the ten genes contained predicted gRNA off-targets within 2 kb of the transcription start site, suggesting that the increased efficacy of ZIM3 KRAB domain does not lead to additional silencing of off-target genes. Additional methods are as described in Example 4.

```
Table of Sequences
dCAS9
SEQ ID NO: 1
MDKKYSIGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPAIKKGI
LQTVKVVDELVKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRSDKNRGKSDNVP
SEEVVKKMKNYWRQLLNAKLITQRKFDNLTKAERGGLSELDKAGFIKRQLVETRQITKHVAQILDSR
MNTKYDENDKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTK
EVLDATLIHQSITGLYETRIDLSQLGGD
KRAB domain of Zim3
(SEQ ID NO: 2)
VTFEDVTVNFTQGEWQRLNPEQRNLYRDVMLENYSNLVSVGQGETTKPDVILRLEQGKEPWL
KRAB domain in SEQ ID NOs: 3-10 and 18 is bolded.
ZIM3 (Q9NZV7)
(SEQ ID NO: 3)
MNNSQGRVTFEDVTVNFTQGEWQRLNPEQRNLYRDVMLENYSNLVSVGQGETTKPDVILRLEQGKEP
WLEEEEVLGSGRAEKNGDIGGQIWKPKDVKESL
ZNF554 (Q86TJ5)
(SEQ ID NO: 4)
MFSQEERMAAGYLPRWSQELVTFEDVSMDFSQEEWELLEPAQKNLYREVMLENYRNVVSLEALKNQC
TDVGIKEGPLSPAQTSQVTSLSSWTGYLLFQPVASSHLEQREALWIEEKGTPQASCSDWMTVLRNQD
STYKKVALQE
ZNF264 (O43296)
(SEQ ID NO: 5)
MAAAVLTDRAQVSVTFDDVAVTFTKEEWGQLDLAQRTLYQEVMLENCGLLVSLGCPVPKAELICHLE
HGQEPWTRKEDLSQDTCPGDKGKPKTTEPTTCEPALSE
ZNF324 (O75467)
(SEQ ID NO: 6)
MAFEDVAVYFSQEEWGLLDTAQRALYRRVMLDNFALVASLGLSTSRPRVVIQLERGEEPWVPSGTDT
TLSRTTYRRRNPGSWSLTEDRDVSG
ZNF669 (Q96BR6)
(SEQ ID NO: 7)
MHFRRPDPCREPLASPIQDSVAFEDVAVNFTQEEWALLDSSQKNLYREVMQETCRNLASVGSQWKDQ
NIEDHFEKPGKDIRNHIVQRLCESKEDGQYGEVVSQIPNLDLNENISTGLKPCECSICGK
ZNF354A (Q60765)
(SEQ ID NO: 8)
MAAGQREARPQVSLTFEDVAVLFTRDEWRKLAPSQRNLYRDVMLENYRNLVSLGLPFTKPKVISLLQ
QGEDPWEVEKDGSGVSSLGSKSSHKTTKSTQTQDSSFQ
ZFP82 (Q8N141)
(SEQ ID NO: 9)
MALRSVMFSDVSIDFSPEEWEYLDLEQKDLYRDVMLENYSNLVSLGCFISKPDVISSLEQGKEPWKV
VRKGRRQYPDLETKYETKKLSLENDIYEIN
ZNF566 (Q969W8)
(SEQ ID NO: 10)
MAQESVMFSDVSVDFSQEEWECLNDDQRDLYRDVMLENYSNLVSMGHSISKPNVISYLEQGKEPWLA
DRELTRGQWPVLESRCETKKLFLKKEIYEIESTQWEIMEK
ZIM2 (Q9NZV7)
(SEQ ID NO: 18)
MAGSQFPDFKHLGTFLVFEELVTFEDVLVDFSPEELSSLSAAQRNLYREVMLENYRNLVSLGHQFSK
PDIISRLEEEESYAMETDSRHTVICQGE
Tracr sequences
(SEQ ID NO: 11)
5′-
gtttcagagctatgctggaaacagcatagcaagttgaaataaggctagtccgttatcaacttgaaaaagtggcaccgagtcggt
gc-3′
(SEQ ID NO: 12)
5′-GTTTTAGAGCTAGAAATAGCAAGTTAAAATAAGGCTAGTCCGTTATCAACTTGAAAAAG
TGGCACCGAGTCGGTGC-3′
tracr-v2
(SEQ ID NO: 13)
5′-GTTTCAGAGCTACAGCAGAAATGCTGTAGCAAGTTGAAAT-3′
Promoter and enhancer used for fusion construct ZIM3-KRAB -dCAS9 HA TAGGED
CMV promoter + enhancer
(SEQ ID NO: 15)
CGTTACATAACTTACGGTAAATGGCCCGCCTGGCTGACCGCCCAACGACCCCCGCCCATTGACGTCA
ATAATGACGTATGTTCCCATAGTAACGCCAATAGGGACTTTCCATTGACGTCAATGGGTGGAGTATT
TACGGTAAACTGCCCACTTGGCAGTACATCAAGTGTATCATATGCCAAGTACGCCCCCTATTGACGT
CAATGACGGTAAATGGCCCGCCTGGCATTATGCCCAGTACATGACCTTATGGGACTTTCCTACTTGG
CAGTACATCTACGTATTAGTCATCGCTATTACCATGGTGATGCGGTTTTGGCAGTACATCAATGGGC
GTGGATAGCGGTTTGACTCACGGGGATTTCCAAGTCTCCACCCCATTGACGTCAATGGGAGTTTGTT
TTGGCACCAAAATCAACGGGACTTTCCAAAATGTCGTAACAACTCCGCCCCATTGACGCAAATGGGC
GGTAGGCGTGTACGGTGGGAGGTCTATATAAGCAGAGCTCTCTGGCTAACTGTCGGGATCA
Fusion construct ZIM3-KRAB -dCAS9 HA TAGGED
                  ZIM3-KRAB dCas9 HA NLS, STOP*
(SEQ ID NO: 14)

[symbol]

[symbol]

[symbol]

[symbol]

[symbol]

[symbol]

[symbol]

              GGSGGSPKKKRKVGRVC
RISSLRYRGPGIATMDKKYSIGLAIGTNSVGWAVITDEYKVPSKKFKVLGNTDRHSIKKNLIGALLF
DSGETAEATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGLFGNLIALSLGLTPNFKS
NFDLAEDAKLQLSKDTYDDDLDNLLAQIGDQYADLFLAAKNLSDAILLSDILRVNTEITKAPLSASM
IKRYDEHHQDLTLLKALVRQQLPEKYKEIFFDQSKNGYAGYIDGGASQEEFYKFIKPILEKMDGTEE
LLVKLNREDLLRKQRTFDNGSIPHQIHLGELHAILRRQEDFYPFLKDNREKIEKILTFRIPYYVGPL
ARGNSRFAWMTRKSEETITPWNFEEVVDKGASAQSFIERMTNFDKNLPNEKVLPKHSLLYEYFTVYN
ELTKVKYVTEGMRKPAFLSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTYAHLFDDKVMKQLKRRRY
TGWGRLSRKLINGIRDKQSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKGQKNSRERMKRIEEGIKE
LGSQILKEHPVENTQLQNEKLYLYYLQNGRDMYVDQELDINRLSDYDVDAIVPQSFLKDDSIDNKVL
TRSDKNRGKSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKTEITLANGEIR
KRPLIETNGETGEIVWDKGRDFATVRKVLSMPQVNIVKKTEVQTGGFSKESILPKRNSDKLIARKKD
WDPKKYGGFDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPEDNEQKQLFV
EQHKHYLDEIIEQISEFSKRVILADANLDKVLSAYNKHRDKPIREQAENIIHLFTLTNLGAPAAFKY
FDTTIDRKRYTSTKEVLDATLIHQSITGLYETRIDLSQLGGDAYPYDVPDYASLGSGS [symbol] ED [symbol]
DG*
Fusion construct ZIM3-KRAB-PYL1
ZIM3-KRAB PYL1, STOP*
(SEQ ID NO: 16)

[symbol]

[symbol]

[symbol]

[symbol]

[symbol]

[symbol]

[symbol]

              DIQHSGGRSSGSGSTSG
SGKTGGGGAPTQDEFTQLSQSIAEFHTYQLGNGRCSSLLAQRIHAPPETVWSVVRRFDRPQIYKHFI
KSCNVSEDFEMRVGCTRDVNVISGLPANTSRERLDLLDDDRRVTGFSITGGEHRLRNYKSVTTVHRF
EKEEEEERIWTVVLESYVVDVPEGNSEEDTRLFADTVIRLNLQKLASITEAMN
-dCas9-mCherry-ZIM3-KRAB
dCas9, HA, NLS, , ZIM3-KRAB STOP*
(SEQ ID NO: 17)
MDKKYSIGLAIGTNSVGWAVITDEYKVPSKKFKVLGNTDRHSIKKNLIGALLFDSGETAEATRLKRT
ARRRYTRRKNRICYLQEIFSNEMAKVDDSFFHRLEESFLVEEDKKHERHPIFGNIVDEVAYHEKYPT
IYHLRKKLVDSTDKADLRLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRFAWMTRKS
EETITPWNFEEVVDKGASAQSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVEDRFNASLGTYHDLLKIIKD
KDFLDNEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPAIKKGI
LQTVKVVDELVKVMGRHKPENIVIEMARENQTTQKGQKNSRERMKRIEEGIKELGSQILKEHPVENT
QLQNEKLYLYYLQNGRDMYVDQELDINRLSDYDVDAIVPQSFLKDDSIDNKVLTRSDKNRGKSDNVP
SEEVVKKMKNYWRQLLNAKLITQRKFDNLTKAERGGLSELDKAGFIKRQLVETRQITKHVAQILDSR
MNTKYDENDKLIREVKVITLKSKLVSDFRKDFQFYKVREINNYHHAHDAYLNAVVGTALIKKYPKLE
SEFVYGDYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKKYGGFDSPTV
AYSVLVVAKVEKGKSKKLKSVKELLGITIMERSSFEKNPIDFLEAKGYKEVKKDLIIKLPKYSLFEL
ENGRKRMLASAGELQKGNELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAAFKYFDTTIDRKRYTSTK
EVLDATLIHQSITGLYETRIDLSQLGGDAYPYDVPDYASLGSGSPKKKRKVEDPKKKRKVDGIGSGS
NGSSGS
                                                                              GGGGG

[symbol]

[symbol]

[symbol]

[symbol]

[symbol]

[symbol]

[symbol]

[symbol]


5′-N₁NGG-3′
where N₁ is 15 to 25, 16 to 24, 17 to 23, 18 to 22, or 19 to 21
nucleotides in length, optionally 20 nucleotides in length or any
number between and including 15 and 25
```

### REFERENCES

1. Fulco, C. P. et al. Systematic mapping of functional enhancer-promoter connections with CRISPR interference. Science (New York, NY) 354, 769-773 (2016).
2. Gilbert, L. A. et al. Genome-Scale CRISPR-Mediated Control of Gene Repression and Activation. Cell 159, 647-661 (2014).
3. Kampmann, M. CRISPRi and CRISPRa Screens in Mammalian Cells for Precision Biology and Medicine. ACS Chem. Biol. 13, 406-416 (2018).
4. Qi, L. S. et al. Repurposing CRISPR as an RNA-guided platform for sequence-specific control of gene expression. Cell 152, 1173-1183 (2013).
5. Liu, S. J. et al. CRISPRi-based genome-scale identification of functional long noncoding RNA loci in human cells. Science 355, eaah7111 (2017).
6. Meyers, R. M. et al. Computational correction of copy number effect improves specificity of CRISPR-Cas9 essentiality screens in cancer cells. Nature Genetics 49, 1779-1784 (2017).
7. Horlbeck, M. A. et al. Compact and highly active next-generation libraries for CRISPR-mediated gene repression and activation. eLife 5, e19760 (2016).
8. Yeo, N. C. et al. An enhanced CRISPR repressor for targeted mammalian gene regulation. Nat Methods 15, 611-616 (2018).
9. Sanson, K. R. et al. Optimized libraries for CRISPR-Cas9 genetic screens with multiple modalities. Nat Commun 9, 1-15 (2018).
10. Margolin, J. F. et a/. Kruppel-associated boxes are potent transcriptional repression domains. Proceedings of the National Academy of Sciences 91, 4509-4513 (1994).
11. Helleboid, P. et al. The interactome of KRAB zinc finger proteins reveals the evolutionary history of their functional diversification. EMBO J (2019) doi:10.15252/embj.2018101220.
12. Murphy, K. E. et al. The Transcriptional Repressive Activity of KRAB Zinc Finger Proteins Does Not Correlate with Their Ability to Recruit TRIM28. PLOS ONE 11, e0163555 (2016).
13. Schmitges, F. W. et al. Multiparameter functional diversity of human C2H2 zinc finger proteins. Genome Res. 26, 1742-1752 (2016).
14. Gao, Y. et at. Complex transcriptional modulation with orthogonal and inducible dCas9 regulators. Nature Methods 13, 1043-1049 (2016).
15. Amabile, A. et a/. Inheritable Silencing of Endogenous Genes by Hit-and-Run Targeted Epigenetic Editing. Cell 167, 219-232.e14 (2016).
16. Rosenbluh, J. et al. Complementary information derived from CRISPR Cas9 mediated gene deletion and suppression. Nat Commun 8, 15403 (2017).
17. Ecco, G., Imbeault, M. & Trono, D. KRAB zinc finger proteins. Development (Cambridge, England) 144, 2719-2729 (2017).
18. Naglich, J. G., Metherall, J. E., Russell, D. W. & Eidels, L. Expression cloning of a diphtheria toxin receptor: identity with a heparin-binding EGF-like growth factor precursor. Cell 69, 1051-1061 (1992).
19. Dixit, A. et al. Perturb-Seq: Dissecting Molecular Circuits with Scalable Single-Cell RNA Profiling of Pooled Genetic Screens. Cell 167, 1853-1866.e17 (2016).
20. Norman, T. M. et al. Exploring genetic interaction manifolds constructed from rich single-cell phenotypes. Science 365, 786-793 (2019).
21. Horlbeck, M. A. et al. Mapping the Genetic Landscape of Human Cells. Cell 174, 953-967.e22 (2018).
22. Zhang et al A Multicolor Panel of TALE-KRAB based transcriptional repressor vectors enabling knockdown of multiple gene targets. Sci Rep 4, 7338 (2015) https://doi.org/10.1038/srep07338.
23. Deuschle, U et al Tetracycline-reversible silencing of eukaryotic promoters. MCB 15, 1907 (1995).
24. Kiani S et al. Cas9 gRNA engineering for genome editing, activation and repression. Nature Methods 12, 1051 (2015).
25. Beerli R. R. et al Toward controlling gene expression at will: Specific regulation of the erbB-2/HER-2 promoter by using polydactyl zinc finger proteins constructed from modular building blocks PNAS 95, 14628 (1995).
26. https://www.nature.com/articles/ncomms1962
27. Gilbert L A, Larson M H, Morsut L, Liu Z, Brar G A, Torres S E, Stern-Ginossar N, Brandman O, Whitehead E H, Doudna J A, Lim W A, Weissman J S, Qi L S Cell. 2013 Jul 9. pii: S0092-8674(13)00826-X. doi: 10.1016/j.cell.2013.06.044 https://www.cell.com/fulltext/S0092-8674(13)00826-X Cell. 2013 Jul 9. pii: 50092-8674(13)00826-X. doi: 10. 10164.cell.2013.06.044.
28. https://www.nature.com/articles/s41467-018-07901-8
29. https://www.cell.com/cell/fulltext/S0092-8674(15)01495-

1. A heterologous transcriptional repressor comprising:
a DNA targeting domain, optionally a CRISPR-Cas protein, preferably an enzymatically inactive CRISPR-CAS 9 protein, zinc finger domain, tet-repressor or TALE; and at least one KRAB domain selected from the group consisting of ZIM3-KRAB, ZIM2-KRAB, ZNF554-KRAB, ZNF264-KRAB, ZNF324-KRAB, ZNF354A-KRAB, ZFP82-KRAB, and ZNF669-KRAB.

2. The transcriptional repressor of claim 1, further comprising at least one interaction component.

3. The transcriptional repressor of claim 1, wherein the DNA targeting domain and KRAB domain are domains of a single polypeptide.

4. The transcriptional repressor of claim 2, comprising
a first polypeptide comprising the DNA targeting domain and a first interaction component, and
a second polypeptide comprising a KRAB domain and a second interaction component,
wherein the first and second interaction components interact under suitable conditions.

5. The transcriptional repressor of claim 4, wherein the first and second interaction components form an inducible heterodimer pair which interact under inducing conditions, optionally ABI1 and PYL1.

6. The transcriptional repressor of claim 1, wherein the DNA targeting domain is an enzymatically inactive CRISPR-Cas protein, optionally dCas9 or dCas12a.

7. The transcriptional repressor of claim 1, wherein the at least one KRAB domain is selected from any one of the KRAB domains of SEQ ID Nos: 4-10 or 19, optionally ZIM3-KRAB.

8. The transcriptional repressor of claim 1, further comprising one or more nuclear localization signals (NLS), optionally an SV40 NLS.

9. The transcriptional repressor of claim 1, wherein the transcriptional repressor has an amino acid sequence of SEQ ID NO: 14, 16 or 17, or at least 80%, 85%, 90%, 95% or 99% to the DNA targeting domain and KRAB domains portions thereof.

10. An isolated nucleic acid encoding the transcriptional repressor of claim 1.

11. An expression construct comprising the nucleic acid of claim 10 operably linked to one or more promoters and one or more transcription termination sites.

12. A vector comprising the nucleic acid of claim 10 or an expression construct comprising the nucleic acid, optionally wherein the vector is an adenoviral or lentiviral vector.

13. A cell comprising the transcriptional repressor of claim 1, a nucleic acid encoding the transcriptional repressor, an expression construct comprising the nucleic acid, or a vector comprising the nucleic acid or the expression construct.

14. A transcriptional repression system comprising:
a) the heterologous transcriptional repressor of claim 1, a nucleic acid encoding the transcriptional repressor, an expression construct comprising the nucleic acid, a vector comprising the nucleic acid or the expression construct or a cell comprising the transcriptional repressor, the nucleic acid, the expression construct or the vector, wherein the DNA targeting domain comprises a CRISPR-Cas protein; and optionally
b) at least one gRNA and/or at least one inducing agent.

15. The transcriptional repression system of claim 14, wherein the at least one gRNA targets a regulatory element of a gene, optionally the regulatory element is a promoter region, an enhancer region, or a distal regulatory site.

16. A method of repressing transcription of a target gene in a cell, the method comprising:
a) introducing into the cell the transcriptional repressor of claim 1, a nucleic acid encoding the transcriptional repressor, an expression construct comprising the nucleic acid, or a vector comprising the nucleic acid or the expression construct; and
b) culturing the cell under suitable conditions such that the at least one KRAB domain represses transcription of the target gene.

17. The method of claim 16, wherein the DNA targeting domain comprises a CRISPR-Cas protein, the method further comprises introducing into the cell at least one gRNA, and culturing the cell under suitable conditions such that the at least one gRNA associates with the CRISPR-Cas protein to guide the transcriptional repressor to a CRISPR target site.

18. A screening method, the method comprising:
a) introducing into a plurality of cells the transcriptional repressor of claim 1, a nucleic acid encoding the transcriptional repressor, an expression construct comprising the nucleic acid, or a vector comprising the nucleic acid or the expression construct, wherein the DNA targeting domain comprises a CRISPR-Cas protein; and a plurality of gRNAs; or introducing a plurality of gRNAs into a population of cells comprising the transcriptional repressor, the nucleic acid, the expression construct or the vector, wherein the DNA targeting domain comprises a CRISPR-Cas protein;
b) culturing the plurality of cells such that the one or more gRNAs associate with the CRISPR-Cas protein and guides the transcriptional repressor to a CRISPR target site such that the at least one KRAB domain represses transcription of a target gene;
c) optionally treating with an amount of a test drug or toxin;
d) optionally culturing the plurality of cells for a period of time to allow for gRNA dropout or enrichment; and
e) collecting the plurality of cells, or a subset thereof.

19. The method of claim 18, wherein the method further comprises identifying one or more gRNAs that are over- or under-represented in the plurality of cells or subset thereof.

20. A composition comprising the transcriptional repressor of claim 1, a nucleic acid encoding the transcriptional repressor, an expression construct comprising the nucleic acid, a vector comprising the nucleic acid or the expression construct, or a cell comprising the transcriptional repressor, the nucleic acid, the expression construct or the vector.

21. A kit comprising a vial and the heterologous transcriptional repressor of any one of claim 1, a nucleic acids encoding the transcriptional repressor, an expression construct comprising the nucleic acid, a vector comprising the nucleic acid or the expression construct, a cell comprising the transcriptional repressor, the nucleic acid, the expression construct or the vector, or a composition comprising the transcriptional repressor, the expression construct, the vector or the cell and optionally one or more of: an inducing agent, a gRNA or a gRNA expression construct.


SEQUENCE LISTING (18 sequences)

SEQ ID NO: 1 - protein, 1368 aa, Artificial Sequence
  Synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 62 aa, Homo sapiens
VTFEDVTVNFTQGEWQRLNPEQRNLYRDVMLENYSNLVSVGQGETTKPDVILRLEQGKEPWL

SEQ ID NO: 3 - protein, 100 aa, Homo sapiens
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 4 - protein, 144 aa, Homo sapiens
MFSQEERMAAGYLPRWSQELVTFEDVSMDFSQEEWELLEPAQKNLYREVMLENYRNVVSLEALKNQCTDVGIKEGPLSPAQTSQVTSLSSWTGYLLFQPVASSHLEQREALWIEEKGTPQASCSDWMTVLRNQDSTYKKVALQE

SEQ ID NO: 5 - protein, 105 aa, Homo sapiens
MAAAVLTDRAQVSVTFDDVAVTFTKEEWGQLDLAQRTLYQEVMLENCGLLVSLGCPVPKAELICHLEHGQEPWTRKEDLSQDTCPGDKGKPKTTEPTTCEPALSE

SEQ ID NO: 6 - protein, 92 aa, Homo sapiens
MAFEDVAVYFSQEEWGLLDTAQRALYRRVMLDNFALVASLGLSTSRPRVVIQLERGEEPWVPSGTDTTLSRTTYRRRNPGSWSLTEDRDVSG

SEQ ID NO: 7 - protein, 127 aa, Homo sapiens
MHFRRPDPCREPLASPIQDSVAFEDVAVNFTQEEWALLDSSQKNLYREVMQETCRNLASVGSQWKDQNIEDHFEKPGKDIRNHIVQRLCESKEDGQYGEVVSQIPNLDLNENISTGLKPCECSICGK

SEQ ID NO: 8 - protein, 105 aa, Homo sapiens
MAAGQREARPQVSLTFEDVAVLFTRDEWRKLAPSQRNLYRDVMLENYRNLVSLGLPFTKPKVISLLQQGEDPWEVEKDGSGVSSLGSKSSHKTTKSTQTQDSSFQ

SEQ ID NO: 9 - protein, 97 aa, Homo sapiens
MALRSVMFSDVSIDFSPEEWEYLDLEQKDLYRDVMLENYSNLVSLGCFISKPDVISSLEQGKEPWKVVRKGRRQYPDLETKYETKKLSLENDIYEIN

SEQ ID NO: 10 - protein, 107 aa, Homo sapiens
MAQESVMFSDVSVDFSQEEWECLNDDQRDLYRDVMLENYSNLVSMGHSISKPNVISYLEQGKEPWLADRELTRGQWPVLESRCETKKLFLKKEIYEIESTQWEIMEK

SEQ ID NO: 11 - DNA, 86 bp, Artificial Sequence
  Synthetic construct
gtttcagagctatgctggaaacagcatagcaagttgaaataaggctagtccgttatcaacttgaaaaagtggcaccgagtcggtgc

SEQ ID NO: 12 - DNA, 76 bp, Artificial Sequence
  Synthetic construct
gttttagagctagaaatagcaagttaaaataaggctagtccgttatcaacttgaaaaagtggcaccgagtcggtgc

SEQ ID NO: 13 - DNA, 40 bp, Artificial Sequence
  Synthetic construct
gtttcagagctacagcagaaatgctgtagcaagttgaaat

SEQ ID NO: 14 - protein, 1550 aa, Artificial Sequence
  Synthetic construct
  MISC_FEATURE (1)..(8): Flanking
  MISC_FEATURE (9)..(108): ZIM3-KRAB
  MISC_FEATURE (109)..(117): Flanking
  MISC_FEATURE (149)..(1516): dCas9
  MISC_FEATURE (1518)..(1526): HA
  MISC_FEATURE (1533)..(1539): NLS
  MISC_FEATURE (1542)..(1548): NLS
TSLYKKVGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLYPTFLYKVVGGSGGSPKKKRKVGRVCRISSLRYRGPGIATMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDG

SEQ ID NO: 15 - DNA, 554 bp, Artificial Sequence
  Synthetic construct
  misc_feature (531)..(554): Flanking
  misc_feature: CMV promoter + enhancer
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactgtcgggatcaacaagtttgtacaaaaaagttggc

SEQ ID NO: 16 - protein, 321 aa, Artificial Sequence
  Synthetic construct
  MISC_FEATURE (1)..(8): Flanking
  MISC_FEATURE (9)..(108): ZIM3-KRAB
  MISC_FEATURE (109)..(117): Flanking
  MISC_FEATURE (140)..(321): PYL1
TSLYKKVGMNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLYPTFLYKVVDIQHSGGRSSGSGSTSGSGKTGGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 17 - protein, 1749 aa, Artificial Sequence
  Synthetic construct
  MISC_FEATURE (1)..(1368): dCas9
  MISC_FEATURE (1370)..(1378): HA
  MISC_FEATURE (1385)..(1391): NLS
  MISC_FEATURE (1394)..(1400): NLS
  MISC_FEATURE (1414)..(1648): mCherry
  MISC_FEATURE (1654)..(1749): ZIM3-KRAB
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYGGGGGMGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESL

SEQ ID NO: 18 - protein, 95 aa, Homo sapiens
MAGSQFPDFKHLGTFLVFEELVTFEDVLVDFSPEELSSLSAAQRNLYREVMLENYRNLVSLGHQFSKPDIISRLEEEESYAMETDSRHTVICQGE